SEQUENCE LISTING (64 sequences)

SEQ ID NO: 1 - DNA, 31 bp, Homo sapiens
  variation (16)..(16): n= G or A
caatagaaaacttaangccgcaactaagtaa

SEQ ID NO: 2 - DNA, 31 bp, Homo sapiens
  variation (16)..(16): n= C or T
ctaaggcccctcagtntctctgtgcctctgg

SEQ ID NO: 3 - DNA, 31 bp, Homo sapiens
  variation (16)..(16): n= T or C
atggtgagtttgcatnaattttgaaaagaaa

SEQ ID NO: 4 - DNA, 31 bp, Homo sapiens
  variation (16)..(16): n= T or G
ctagagtcaagtaagngtttctgtctctaac

SEQ ID NO: 5 - DNA, 31 bp, Homo sapiens
  variation (16)..(16): n= G or A
cagacataggcatggnttagtcctgttgcca

SEQ ID NO: 6 - DNA, 31 bp, Homo sapiens
  variation (16)..(16): n= T or C
ctataagctctgttanacacttggccaaggt

SEQ ID NO: 7 - DNA, 31 bp, Homo sapiens
  variation (16)..(16): n= G or A
ttagaagaggcagacntaatccagtgcattt

SEQ ID NO: 8 - DNA, 31 bp, Homo sapiens
  variation (16)..(16): n= C or T
ttcacacacaaagacncataaagttgtacgt

SEQ ID NO: 9 - DNA, 31 bp, Homo sapiens
  variation (16)..(16): n= C or T
taaaatcagagtagcnaatgagagctaaagt

SEQ ID NO: 10 - DNA, 31 bp, Homo sapiens
  variation (16)..(16): n= C or G
ccataattttgactcngaattacaaagtatt

SEQ ID NO: 11 - DNA, 31 bp, Homo sapiens
  variation (16)..(16): n= C or T
agcaacagtttctaangggcaaataatttaa

SEQ ID NO: 12 - DNA, 31 bp, Homo sapiens
  variation (16)..(16): n= G or A
ataaatggaaagaatnagaacaatgggaaaa

SEQ ID NO: 13 - DNA, 31 bp, Homo sapiens
  variation (16)..(16): n= C or T
cttgagtcatcaggcnatagtcctaggactg

SEQ ID NO: 14 - DNA, 31 bp, Homo sapiens
  variation (16)..(16): n= C or T
tataagttttctgttnagaaaggccctgaca

SEQ ID NO: 15 - DNA, 31 bp, Homo sapiens
  variation (16)..(16): n= A or G
ttcatgtcttagtgantccacaattgattgt

SEQ ID NO: 16 - DNA, 31 bp, Homo sapiens
  variation (16)..(16): n= G or T
ggtgtttacttctaanatacaatgggagtat

SEQ ID NO: 17 - DNA, 61 bp, Homo sapiens
  variation (31)..(31): n= A or G
atgaaatccctaaggtcttcaagcaatctcntgacaattttttaaaattcacaaaagtacc

SEQ ID NO: 18 - DNA, 61 bp, Homo sapiens
  variation (31)..(31): n= A or G
agacagctgtggaactggaaattaccaaacnatttccaattttaagagatttggatcaggt

SEQ ID NO: 19 - DNA, 31 bp, Homo sapiens
  variation (16)..(16): n= C or A
catttctttgaaaatnaatgtggtttcacca

SEQ ID NO: 20 - DNA, 31 bp, Homo sapiens
  variation (16)..(16): n= A or T
ctgattacagaagcantgcgtgatttttgta

SEQ ID NO: 21 - DNA, 31 bp, Homo sapiens
  variation (16)..(16): n= C or T
agaatcagtgagatanggtatttctgtacat

SEQ ID NO: 22 - DNA, 31 bp, Homo sapiens
  variation (16)..(16): n= G or A
ttggaagctgcgctcnattctattcgctaca

SEQ ID NO: 23 - DNA, 61 bp, Homo sapiens
  variation (31)..(31): n= A or G
ctcgcagcagttgtacttgatgtcgtgtttntagcctggggctttgatgatggcccactcg

SEQ ID NO: 24 - DNA, 31 bp, Homo sapiens
  variation (16)..(16): n= A or G
gctagtttgcccccantggtgccctgctgag

SEQ ID NO: 25 - DNA, 31 bp, Homo sapiens
  variation (16)..(16): n= C or T
aataagtcttgtaagntatatcttctaaagc

SEQ ID NO: 26 - DNA, 6367 bp, Homo sapiens
agacccggggctggctctgctgctctcgcgatatttgcgcgagcctgcttccttctttcctcccttgccagtccgcctgtcttcctccccgtcttccctgcccggcctcccccttcttcccccgctggccccctccccggagggataatatggtctccggcgatggacgccccaaaagcaggatacgcctttgagtaccttattgaaacattaaatgacagttcacataagaagttcttcgatgtatctaaacttggcaccaagtatgatgttctgccttactcaatacgggtcttgttggaagctgctgtacgaaattgtgatggctttttaatgaagaaggaagatgttatgaacattttagactggaaaaccaaacaaagcaatgttgaagtgccctttttccctgcccgtgttcttcttcaagattttactggaataccagcaatggtggattttgctgctatgagggaggcagtgaaaactcttggaggtgatcctgagaaagtccatcctgcttgtccgacagatcttacagttgaccattctttacaaattgacttcagtaaatgtgcaatacagaatgcaccaaatcctggaggtggtgacctgcagaaagcaggaaagctctctccacttaaagtgcagcctaagaagcttccctgcagaggccagactacctgccgaggatcttgtgattctggagaactaggccgaaactcaggaacattttcttcgcagattgagaatacacccatcctgtgtccttttcatttgcaaccagtgcctgaacctgaaacagtgttaaaaaatcaagaagtagaattcggcagaaatcgagagaggcttcagttttttaagtggagttcaagagtttttaagaatgtggcagtgatccctcctggaactggaatggctcatcaaataaacttagaatatttgtcaagagtggtttttgaagaaaaagacctcctcttcccagacagtgtagtcggcacagattcacacataacgatggtgaatggtttagggattctggggtggggggttggaggcattgaaacagaagcagttatgcttggtctgccagtttctcttactttaccagaggtggttggatgtgagttaactgggtcatcaaacccttttgttacatccatagatgttgttcttggtattacaaagcacctcaggcaagtaggagtggctggaaagtttgttgagttttttggaagtggagtttcacaattatctatagttgatcgaactacaatagcaaacatgtgtccggaatatggtgctatcctcagctttttccctgttgacaatgtgacattaaaacatttagaacatacaggttttagcaaagccaaactcgaatcaatggaaacataccttaaagctgtgaaattgtttcgaaatgaccagaattcttcaggagaacctgaatactcccaggtgatccagattaatctgaattcaatagttccatctgttagtggtccaaaaagacctcaggatagagttgctgtgacagatatgaaaagcgatttccaggcttgcttaaatgaaaaggttggatttaaaggcttccaaattgcagctgaaaaacaaaaggatattgtctccattcattatgaaggaagtgaatataagctgtctcatggatcagtggtcattgctgcagttatcagttgtaccaataattgcaatccatctgtcatgcttgctgcaggtcttttggctaaaaaggctgttgaagctggtctgcgtgttaaaccttatataagaacaagtttatctccaggcagtgggatggttacacattacctcagttcaagtggagtattaccatatctaagtaagcttggatttgaaatcgttggctatggatgttcaacttgtgtgggaaatacagcacccttatcagacgcagttttaaatgcagtaaaacagggtgatttggttacctgtggaattttatctggaaacaaaaattttgaaggtcgtctttgtgattgtgttcgtgccaattatcttgcctctccacccttagtggtagcttatgccatagcaggcacagtgaatatagatttccagacagaacctttaggtactgaccccaccggcaagaacatttacctgcatgatatttggcctagtcgagaagaagttcatcgagtagaggaagaacatgttatactatccatgtttaaagcattaaaagataaaatagaaatggggaataaacggtggaattccttagaagcaccggattcagttttgtttccatgggacttaaagtctacttatatcagatgcccttcattttttgataaacttaccaaagagccaattgcactccaggctattgaaaatgcccatgtcttattatatttgggagactctgtcacaacagatcatatatcacctgcaggaagtatcgctaggaatagtgctgccgctaagtatttgacaaacagaggccttacccctcgtgaattcaactcttacggagctcgaagaggtaatgatgctgtaatgacaagaggcacttttgcaaatatcaagctttttaataagtttattggaaaaccagctcctaaaacaattcattttccatcaggacagacgctagatgtatttgaggctgcagagctgtaccagaaagaaggtatcccactgattattttagcaggaaagaaatatggttcaggaaactccagagactgggctgccaaaggaccgtatttactgggtgtgaaagctgttttggccgaaagttatgaaaaaatacacaaagatcatttgattggaattggcatagctccacttcagttccttccaggagaaaatgcagattccttgggcctctccggtagagaaacattttctttaacatttcctgaagaactgtctcctggaattacattgaatatacagacaagcactggaaaagtattcagcgtgattgcttcgtttgaagatgatgtggaaataacattatacaaacatggaggattattaaactttgtggcacgaaaattctcatagtatctacttaccatagatacctttcataactggtaactgcaaagccttttgtgctggacccaggaatccttaccatggagcagcagatagtcccagtatactcacttatctcatccatggatgtaaatgatgatgaatcaacatagtaactgaaatgaaatcttcttgattttaaataatatacgaatggtgctattaatattgctaaaatcaacgtgtgaagtgtgttgtggaagagacctgtaagtatggggggggggcgatattttatcagaccattttgtaaataaaggcagaatttgtgttgaagattcttatatgaataaccttcctggatttgtttagttttgcaccaataaaactgtgtactactgtttgttggtttaagagtagcagattgaaatataagaagccagattagactctaaaattgtggccattggaatctcatttataaatggaccttttaagtatattaattcctcttcagaattgagctggacacatttggcattcttagtttgtcatataaccaggtttatccttagtctaactgcaagggatagaacctgccccaggtcacaatcattctgtccaatccagccaaggttccctccacatatgaagatggaccatggcaggatacaactgattgtgtggcaccatgtattagcagtgggaatatgtatcacatatgatgcagcctttcatatttcagcagtttgccactgtgactgtctggcaagcccccagatggcgttatattaattggattagattattttgctcaccttatgtaatactgtaacttcctataacctaatattttcggtatcattaaccaaaatttcacactcatagttgctaaagagaatgttattcaatcattaaactctgaactgatttcttctatacatttaaattatccaccctcaataatacgggtgctcaaccctatgcattttttaagtgttgctatttcttaattaaattgatttcctgtcattttgaatcatttatcacctgcgatgcatgattctattaattttgttatgttactgttttaaccaaagctgaccgtaaggataaaacacttaagttgttgctgagtactatatatcctcaatatgcatgtctgcccatcacatcaaatgttctgtcaacaagatgtttggtaattttttttaaaaaggttggaaaattaattatagaaggttctatactgtttttttaattaagaaactaaatctagcaggctaaaggttaattgtagtgattttttttcacatagatatctttctatgacctagttagttactgcaattcagaattagttcacattgcataaagaattacttgttgtaagcaaaatgctgaaactaccaaaccagtggatgaagaccactaagaactttgcacataatcatacaatcttttgaaaatattttgcaaatatgtgtttagacaataagatggactagagttcgacaaaatgatttctttatttaaattttgtataagtattttcttcgacactttcaaattatattgtgttcttgatatatgctgtatatttatttgttagtgcatgtgtttttaatttacatgaaaacatgagttaggagaaattacaggttgaaagatgaaatgcctgtatgtgctctgaagaaatggtaattccagattgtgcagggggaaacaaatctattttgttttgttttgttttttgagacggagtcttgccctgtggctgggctggagtgcaatggcgcgatcttggctcactgcaacctccgcctcccgggttcaggggattctcctgcctcagcctcctgagtagctgggactacaagcatgcaccaccacgcccagctaatttttgtagttttagtagagatggggtttcaccatgttggccagatgatctgaatctcttgacctcgtgatccgcccgccttggcctccacaagtgctgggattacaggtgtgagccaccacacccggcctatattgttttgaaagcatactctatatatagttatgggcagaggcacaggcatcctcagcagctgattcaggagatgatggtaaagctagctaactatgaattaaacattcacatatccagtctacctggtccagtaataatacaagcaaatcttgtatttcaggaacaaatcaaggttctcttaattttttggcttatatacaatgaagtaaaaacttgataaacatggtttcaaattgaggaggagagtcttggatgtatgttttaatatgtataccttataattctgcctctagccaaatgctatgtttgcaaaatgtggcatctgttagtttttattgtctgtgtcttctttgtttactataccttgggtaattttgtgttaccaaaaaaaaaaaaaaaaaaaggaagtgtaatgtcagacacacaagaaaagcaaatcagtgttgtaagcttaaagtacaatttcaaaggtcactaccaacagcagggttttttttatactttgaaaacattatgctacatatcattgccattttcatattttggggttttgctactcttatacaatggaatcaatggaaatgtcatccagccactgaattgccattattatatctaaaaagtttctaagatgacagttatcactattttgttttatctccatgctgacatttgaaagaaggtactagtatccctctagccagattgcttagtttttcgttggtaatcaaacaacagttgtactaaaggaaagtaaagctaggacctaaatcagaatcatagttgcctgcatatatggtaacaaggtcgtgtgcatttgctttcacagtgatgagtgagaggatgagaagaaattatttgacatttttctgtggttgaatagaagacacctttcttttgtctttaggtttaggaggagatactaagatactggatgtttatcctatcttagtttggttggagtaataagagagaagaagagggtggactttggcttttcagtgttttttcccctaaagagtgatattgctgacgtttctatcaattttacacataatatgtggctatgaaaccatatatctcacttaagtaacaaagtaatcactttgtctatcactaagtaatagacaaaaatcattgtctattatttaaagccaacaaaacagtgtaacagttttaagttcaataatgttaagtattgtatagaaatatattggaggcaaagttcagttgatgacaattgtgtatatgttactgatgctgtaaattatttttaataaagaaaattgtattatcacattt

SEQ ID NO: 27 - DNA, 1235 bp, Homo sapiens
gcgggcgctgcggcccctgctctacctcctagcgccggtgcgcggccgaggccgcactacctgtctgcgggaaagcgggatccaccccaggacgtcgggtcgctgccgacataatgtcaagtggaaactatcagcagtcagaggctcttagcaaacccactttcagtgaggaacaagcctctgcgttagtggagtcagtgtttgggttgaaagtttccaaggtccggccacttcctagctatgatgaccaaaactttcatgtctacgtttcaaaaaccaaagatggcccaactgaatatgtcctcaaaataagcaacaccaaggctagcaaaaatccagacctgattgaagtgcagaatcacatcatcatgtttctgaaagccgctggatttccaacagcctctgtgtgtcacactaaaggagacaacacagcttctctcgtgtctgtagatagtggctctgaaatcaaaagctacttggtgaggctgctgacttacctcccaggaagacccatcgctgagcttcccgtcagcccccagctattgtatgaaattggaaaactagctgccaaattggataagacactgcagagattccatcacccaaagttaagtagtcttcatcgggagaacttcatctggaatctgaaaaatgttcctcttctggagaaatacctgtatgccctgggccagaatcgaaaccgagagattgttgagcatgtcattcatctgttcaaggaggaagtaatgaccaaattaagtcattttcgagaatgtatcaatcacggagatcttaatgaccataatattttaatagagtccagcaagtcagcctctggaaatgctgaatatcaagtgtctgggattttagactttggtgacatgagctatggctactatgtgtttgaagtggcaattaccatcatgtacatgatgattgagagcaagagtcctatacaagtaggaggccatgtccttgcagggtttgaaagcatcaccccactgacagctgtagagaagggtgctttgtttttacttgtatgcagtcgtttttgtcagtcacttgtcatggctgcatactcttgccagctatacccagagaacaaagactatctcatggttactgcaaaaaccgggtggaaacacttacagcaaatgtttgacatgggtcagaaagctgtagaagaaatctggtttgaaactgccaaatcctatgaatctgggatctccatgtga

SEQ ID NO: 28 - DNA, 866 bp, Homo sapiens
gcgggcgctgcggcccctgctctacctcctagcgccggtgcgcggccgaggccgcactacctgtctgcgggaaagcgggatccaccccaggacgtcgggtcgctgccgacataatgtcaagtggaaactatcagcagtcagaggctcttagcaaacccactttcagtgaggaacaagcctctgcgttagtggagtcagtgtttgggttgaaagtttccaaggtccggccacttcctagctatgatgaccaaaactttcatgtctacgtttcaaaaaccaaagatggcccaactgaatatgtcctcaaaataagcaacaccaaggctagcaaaaatccagacctgattgaagtgcagaatcacatcatcatgtttctgaaagccgctggatttccaacagcctctgtgtgtcacactaaaggagacaacacagcttctctcgtgtctgtagatagtggctctgaaatcaaaagctacttggtgaggctgctgacttacctcccaggaagacccatcgctgagcttcccgtcagcccccagctattgtatgaaattggaaaactagctgccaaattggataagacactgcagagattccatcacccaaagttaagtagtcttcatcgggagaacttcatctggaatctgaaaaatgttcctcttctggagaaatacctgtatgccctgggccagaatcgaaaccgagagattgttgagcatgtcattcatctgttcaaggaggaagtaatgaccaaattaagtcattttcgagaatgacctagcaccgtgccaggcccctagaagacccagtaaagatctgttgaataaactgtaagaatgaacacaccaaaaaaaaaaaaaaaaaaa

SEQ ID NO: 29 - DNA, 1189 bp, Homo sapiens
gcgcatgcgcgggggccatattagcagcggttattcggtgagcggtggtggtttattcttccgtggagttaagggctccgtggacatctcaggtcttcagggtcttccatctggaactatataaagttcagaaaacatgtctcgaagatatgactccaggaccactatattttctccagaaggtcgcttataccaagttgaatatgccatggaagctattggacatgcaggcacctgtttgggaattttagcaaatgatggtgttttgcttgcagcagagagacgcaacatccacaagcttcttgatgaagtctttttttctgaaaaaatttataaactcaatgaggacatggcttgcagtgtggcaggcataacttctgatgctaatgttctgactaatgaactaaggctcattgctcaaaggtatttattacagtatcaggagccaataccttgtgagcagttggttacagcgctgtgtgatatcaaacaagcttatacacaatttggaggaaaacgtccctttggtgtttcattgctgtacattggctgggataagcactatggctttcagctctatcagagtgaccctagtggaaattacgggggatggaaggccacatgcattggaaataatagcgctgcagctgtgtcaatgttgaaacaagactataaagaaggagaaatgaccttgaagtcagcacttgctttagctatcaaagtactaaataagaccatggatgttagtaaactctctgctgaaaaagtggaaattgcaacactaacaagagagaatggaaagacagtaatcagagttctcaaacaaaaagaagtggagcagttgatcaaaaaacatgaggaagaagaagccaaagctgagcgtgagaagaaagaaaaagaacagaaagaaaaggataaatagaatcagagattttattactcatttggggcaccatttcagtgtaaaagcagtcctactcttccacactaggaaggctttactttttttaactggtgcagtgggaaaataggacattacatactgaattgggtccttgtcatttctgtccaattgaatactttattgtaacgatgatggttacccttcatggacgtcttaatcttccacacacatcccctttttttggaataaaatttggaaaatggaaatgaaaaaaaaaaaaaaaaa

SEQ ID NO: 30 - DNA, 2468 bp, Homo sapiens
ccacgcgtccgggcgggagctgtggcgcggagcggcccctctgctgcgtctgccctcgttttgtctcacgactcacactcagtgctccattccccaagagttcgcgttccccgcgcggcggtcgagaggcggctgcccgcggtcccgcgcgggcgcggggcgatggcggcgcgggggtcagggccccgcgcgctccgcctgctgctcttggtccagctggtcgcggggcgctgcggtctagcgggcgcggcgggcggcgcgcagagaggattatctgaaccttcttctattgcaaaacatgaagatagtttgcttaaggatttatttcaagactacgaaagatgggttcgtcctgtggaacacctgaatgacaaaataaaaataaaatttggacttgcaatatctcaattggtggatgtggatgagaaaaatcagttaatgacaacaaacgtctggttgaaacaggaatggatagatgtaaaattaagatggaaccctgatgactatggtggaataaaagttatacgtgttccttcagactctgtctggacaccagacatcgttttgtttgataatgcagatggacgttttgaagggaccagtacgaaaacagtcatcaggtacaatggcactgtcacctggactccaccggcaaactacaaaagttcctgtaccatagatgtcacgtttttcccatttgaccttcagaactgttccatgaaatttggttcttggacttatgatggatcacaggttgatataattctagaggaccaagatgtagacaagagagatttttttgataatggagaatgggagattgtgagtgcaacagggagcaaaggaaacagaaccgacagctgttgctggtatccgtatgtcacttactcatttgtaatcaagcgcctgcctctcttttataccttgttccttataataccctgtattgggctctcatttttaactgtacttgtcttctatcttccttcaaatgaaggtgaaaagatttgtctctgcacttcagtacttgtgtctttgactgtcttccttctggttattgaagagatcataccatcatcttcaaaagtcatacctctaattggagagtatctggtatttaccatgatttttgtgacactgtcaattatggtaaccgtcttcgctatcaacattcatcatcgttcttcctcaacacataatgccatggcgcctttggtccgcaagatatttcttcacacgcttcccaaactgctttgcatgagaagtcatgtagacaggtacttcactcagaaagaggaaactgagagtggtagtggaccaaaatcttctagaaacacattggaagctgcgctcgattctattcgctacattacaagacacatcatgaaggaaaatgatgtccgtgaggttgttgaagattggaaattcatagcccaggttcttgatcggatgtttctgtggacttttcttttcgtttcaattgttggatctcttgggctttttgttcctgttatttataaatgggcaaatatattaataccagttcatattggaaatgcaaataagtgaagcctcccaagggactgaagtatacatttagttaacacacatatatctgatggcacctataaaattatgaaaatgtaagttatgtgttaaatttagtgcaagctttaacagactaagttgctaacctcaatttatgttaacagatgatccatttgaacagttggctgtatgactgaagtaataactgatgagatacatttgatcttgtaaaaatagcaaaatattatctgaactggactagtgaaaaatctagtatttgtatcctggcaaataatactaatttataatccacagtaaagttcatcctttgactgtgctggagaattccagttgtatttgaagactgattttaaaacttttctgcatttggtaaaggtatgtaaactttcctgtactcactgagtaacagctaatctttaatataatattatactgctatatttaaaaagctgactacttgatataattacttaatgtgatgcttgatataataattacttaatgtggccgggcacggtggctcacacctgtaatcccagcactttgggaggtcgaggtgggcgtatcacctgaggttgggagttcgagaccagcctgaccaacgtggagaaaccccgtctctactaaaaatatgaaattagccagggtggtggtgcacacctgtaatcccagctacctgggaggctgcggcaggagaatcgcttgaacccaggtggcggaggttgcggtgagctgagatcacgccattgcactccagcctgggcaacaagagcaaaactcagtctcaaataataataataacaacaacttaatgtgctgctgcttttccataaccaacattttaaaaataaatgaaaaacaggaaaaaaaaaaaaaaaaaaaaaaaaaaaaaaaaaaaa

SEQ ID NO: 31 - DNA, 3020 bp, Homo sapiens
gtcctcccgcgggtccgagggcgctggaaacccagcggcggcgaagcggagaggagccccgcgcgtctccgcccgcacggctccaggtctggggtctgcgctggagccgcgcggggagaggccgtctctgcgaccgccgcgcccgctcccgaccgtccgggtccgcggccagcccggccaccagccatgggctctggcccgctctcgctgcccctggcgctgtcgccgccgcggctgctgctgctgctgctgctgtctctgctgccagtggccagggcctcagaggctgagcaccgtctatttgagcggctgtttgaagattacaatgagatcatccggcctgtagccaacgtgtctgacccagtcatcatccatttcgaggtgtccatgtctcagctggtgaaggtggatgaagtaaaccagatcatggagaccaacctgtggctcaagcaaatctggaatgactacaagctgaagtggaacccctctgactatggtggggcagagttcatgcgtgtccctgcacagaagatctggaagccagacattgtgctgtataacaatgctgttggggatttccaggtggacgacaagaccaaagccttactcaagtacactggggaggtgacttggatacctccggccatctttaagagctcctgtaaaatcgacgtgacctacttcccgtttgattaccaaaactgtaccatgaagttcggttcctggtcctacgataaggcgaaaatcgatctggtcctgatcggctcttccatgaacctcaaggactattgggagagcggcgagtgggccatcatcaaagccccaggctacaaacacgacatcaagtacaactgctgcgaggagatctaccccgacatcacatactcgctgtacatccggcgcctgcccttgttctacaccatcaacctcatcatcccctgcctgctcatctccttcctcactgtgctcgtcttctacctgccctccgactgcggtgagaaggtgaccctgtgcatttctgtcctcctctccctgacggtgtttctcctggtgatcactgagaccatcccttccacctcgctggtcatccccctgattggagagtacctcctgttcaccatgatttttgtaaccttgtccatcgtcatcaccgtcttcgtgctcaacgtgcactacagaaccccgacgacacacacaatgccctcatgggtgaagactgtattcttgaacctgctccccagggtcatgttcatgaccaggccaacaagcaacgagggcaacgctcagaagccgaggcccctctacggtgccgagctctcaaatctgaattgcttcagccgcgcagagtccaaaggctgcaaggagggctacccctgccaggacgggatgtgtggttactgccaccaccgcaggataaaaatctccaatttcagtgctaacctcacgagaagctctagttctgaatctgttgatgctgtgctgtccctctctgctttgtcaccagaaatcaaagaagccatccaaagtgtcaagtatattgctgaaaatatgaaagcacaaaatgaagccaaagagattcaagatgattggaagtatgttgccatggtgattgatcgtatttttctgtgggttttcaccctggtgtgcattctagggacagcaggattgtttctgcaacccctgatggccagggaagatgcataagcactaagctgtgtgcctgcctgggagacttccttgtgtcagggcaggaggaggctgcttcctagtaagaacgtactttctgttatcaagctaccagctttgtttttggcatttcgaggtttacttattttccacttatcttggaatcatgcaaaaaaaaaaatgtcaagagtatttattaccgataaatgaacatttaactagcctttttggtatggtaaagagatgtcaaaatgtgattctatgtgattagtatgctatgctatggaatatacatgtaaaaatgtttccttttagttgttgaaacaaaactggatagaaaaatgctgttcagaaatatgaaaagtcattcagttatcactacagatctcccagtaatttttcttatttagcccataatctctttgaaggtttatactaattcagcaatcccccatcgttacccatttcttaccatgcatttctcgttctttactgggtctaaagggctatgcctccatttcagagagcttcaactacttctcttgcatacttctaaattatactatgagaaatcatgcctagttattcattgttaatataactgtcttagtacaccataaactgggtggattataaacaacagaaacttctcagttttggaggttgggaggtccaaggtcaaggcaccagcaaatttggtgtctggtgagggtcctcttcctcaaagggtgccttctagctgtgtcctcacatgactgaagggactagctatctctgtggggtctattttataagggcactaaccccattcatgagagcagagcccccatggcctaatcacctttccaaggccccaccttctatctaagacaatcacgctgggaataggtttcaacatatgaattgggggaggacacatttggaccacagcatgaacctttagaacagggtttctcagccttagcactacggacattttgggctggataaatatgtgttggtacagaatgggggtatcctgtgcattgtaggatctttagcagtaccctagcctcaactcactagatgccaatgacataccttgcttcttcaccagttatgataaccaagaatgtctccattgttaaatgtccccttaggagcaaaattgcccctggttgagaaacattgctttagacaaattgttaagagtatcatgtactacacttctgaaacttaacgtgatcatcaccactgacagatgattcacagagagagactgtttgaatcttgtctcactagtttttcctgtgcaaaaataaaatggacagaattgcaaaaaaaaaaaaaaaaaa

SEQ ID NO: 32 - DNA, 2447 bp, Homo sapiens
ccgccagcaaacctcgggggccaggaccggcgctcactcgaccgcgcggctcacgggtgccctgtgaccccacagcggacgtcgcggcggctgccacccggccccgccggccatgaggcgcgcgccttccctggtccttttcttcctggtcgccctttgcgggcgcgggaactgccgcgtggccaatgcggaggaaaagctgatggacgaccttctgaacaaaacccgttacaataacctgatccgcccagccaccagctcctcacagctcatctccatcaagctgcagctctccctggcccagcttatcagcgtgaatgagcgagagcagatcatgaccaccaatgtctggctgaaacaggaatggactgattaccgcctgacctggaacagctcccgctacgagggtgtgaacatcctgaggatccctgcaaagcgcatctggttgcctgacatcgtgctttacaacaacgccgacgggacctatgaggtgtctgtctacaccaacttgatagtccggtccaacggcagcgtcctgtggctgccccctgccatctacaagagcgcctgcaagattgaggtgaagtactttcccttcgaccagcagaactgcaccctcaagttccgctcctggacctatgaccacacggagatagacatggtcctcatgacgcccacagccagcatggatgactttactcccagtggtgagtgggacatagtggccctcccagggagaaggacagtgaacccacaagaccccagctacgtggacgtgacttacgacttcatcatcaagcgcaagcctctgttctacaccatcaacctcatcatcccctgcgtgctcaccaccttgctggccatcctcgtcttctacctgccatccgactgcggcgagaagatgacactgtgcatctcagtgctgctggcactgacattcttcctgctgctcatctccaagatcgtgccacccacctccctcgatgtgcctctcatcggcaagtacctcatgttcaccatggtgctggtcaccttctccatcgtcaccagcgtctgtgtgctcaatgtgcaccaccgctcgcccagcacccacaccatggcaccctgggtcaagcgctgcttcctgcacaagctgcctaccttcctcttcatgaagcgccctggccccgacagcagcccggccagagccttcccgcccagcaagtcatgcgtgaccaagcccgaggccaccgccacctccaccagcccctccaacttctatgggaactccatgtactttgtgaaccccgcctctgcagcttccaagtctccagccggctctaccccggtggctatccccagggatttctggctgcggtcctctgggaggttccgacaggatgtgcaggaggcattagaaggtgtcagcttcatcgcccagcacatgaagaatgacgatgaagaccagagtgtcgttgaggactggaagtacgtggctatggtggtggaccggctgttcctgtgggtgttcatgtttgtgtgcgtcctgggcactgtggggctcttcctaccgcccctcttccagacccatgcagcttctgaggggccctacgctgcccagcgtgactgagggccccctgggttgtggggtgagaggatgtgagtggccgggtgggcactttgctgcttctttctgggttgtggccgatgaggcctaagtaaatatgtgagcattggccatcaaccccatcaaaccagcccaagccgtggaacaggcaaggatgggggcctgggctgtcctctctgaatgccttggagggatcccaggaagccccagtaggagggagcttcagacagttcaattctggcctgtcttccttccctgcaccgggcaatggggataaagatgacttcgtagcagcacctactatgcttcaggcatggtgccggcctgcctctccatcaccatctctctccacttccccttgtccagttcctcacacacttctagattcttcccagctcagaggcttggcatttgccatatcatcatctttttttttttcttttaaacggagtcttgctatatcgcccaggctcaagtgattctcctgcctgaacctcccaagtagctgggattacagaaagccgccaccgtgcccagctaatttttgtatttttgttggccaggctgatttcgaactcctgacctcagatgacccacctgcctcggcctcccaaagtgctgggattacaggtgtgagccactatgcctggccttccctatcttctacctgaaccttcttcctcttcccccagggcttcccaagctctttccacagccacatcagtcagggcatggctccaatccattctttgctccaatgtcacctcctctgagaggccttccctaaccacccaatcattctaaagcagcctccctacagtcacgttaccctgcctccttc

SEQ ID NO: 33 - protein, 963 aa, Homo sapiens
MDAPKAGYAFEYLIETLNDSSHKKFFDVSKLGTKYDVLPYSIRVLLEAAVRNCDGFLMKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVFKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPQDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSTCVGNTAPLSDAVLNAVKQGDLVTCGILSGNKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 34 - protein, 373 aa, Homo sapiens
MSSGNYQQSEALSKPTFSEEQASALVESVFGLKVSKVRPLPSYDDQNFHVYVSKTKDGPTEYVLKISNTKASKNPDLIEVQNHIIMFLKAAGFPTASVCHTKGDNTASLVSVDSGSEIKSYLVRLLTYLPGRPIAELPVSPQLLYEIGKLAAKLDKTLQRFHHPKLSSLHRENFIWNLKNVPLLEKYLYALGQNRNREIVEHVIHLFKEEVMTKLSHFRECINHGDLNDHNILIESSKSASGNAEYQVSGILDFGDMSYGYYVFEVAITIMYMMIESKSPIQVGGHVLAGFESITPLTAVEKGALFLLVCSRFCQSLVMAAYSCQLYPENKDYLMVTAKTGWKHLQQMFDMGQKAVEEIWFETAKSYESGISM

SEQ ID NO: 35 - protein, 220 aa, Homo sapiens
MSSGNYQQSEALSKPTFSEEQASALVESVFGLKVSKVRPLPSYDDQNFHVYVSKTKDGPTEYVLKISNTKASKNPDLIEVQNHIIMFLKAAGFPTASVCHTKGDNTASLVSVDSGSEIKSYLVRLLTYLPGRPIAELPVSPQLLYEIGKLAAKLDKTLQRFHHPKLSSLHRENFIWNLKNVPLLEKYLYALGQNRNREIVEHVIHLFKEEVMTKLSHFRE

SEQ ID NO: 36 - protein, 261 aa, Homo sapiens
MSRRYDSRTTIFSPEGRLYQVEYAMEAIGHAGTCLGILANDGVLLAAERRNIHKLLDEVFFSEKIYKLNEDMACSVAGITSDANVLTNELRLIAQRYLLQYQEPIPCEQLVTALCDIKQAYTQFGGKRPFGVSLLYIGWDKHYGFQLYQSDPSGNYGGWKATCIGNNSAAAVSMLKQDYKEGEMTLKSALALAIKVLNKTMDVSKLSAEKVEIATLTRENGKTVIRVLKQKEVEQLIKKHEEEEAKAEREKKEKEQKEKDK

SEQ ID NO: 37 - protein, 468 aa, Homo sapiens
MAARGSGPRALRLLLLVQLVAGRCGLAGAAGGAQRGLSEPSSIAKHEDSLLKDLFQDYERWVRPVEHLNDKIKIKFGLAISQLVDVDEKNQLMTTNVWLKQEWIDVKLRWNPDDYGGIKVIRVPSDSVWTPDIVLFDNADGRFEGTSTKTVIRYNGTVTWTPPANYKSSCTIDVTFFPFDLQNCSMKFGSWTYDGSQVDIILEDQDVDKRDFFDNGEWEIVSATGSKGNRTDSCCWYPYVTYSFVIKRLPLFYTLFLIIPCIGLSFLTVLVFYLPSNEGEKICLCTSVLVSLTVFLLVIEEIIPSSSKVIPLIGEYLVFTMIFVTLSIMVTVFAINIHHRSSSTHNAMAPLVRKIFLHTLPKLLCMRSHVDRYFTQKEETESGSGPKSSRNTLEAALDSIRYITRHIMKENDVREVVEDWKFIAQVLDRMFLWTFLFVSIVGSLGLFVPVIYKWANILIPVHIGNANK

SEQ ID NO: 38 - protein, 505 aa, Homo sapiens
MGSGPLSLPLALSPPRLLLLLLLSLLPVARASEAEHRLFERLFEDYNEIIRPVANVSDPVIIHFEVSMSQLVKVDEVNQIMETNLWLKQIWNDYKLKWNPSDYGGAEFMRVPAQKIWKPDIVLYNNAVGDFQVDDKTKALLKYTGEVTWIPPAIFKSSCKIDVTYFPFDYQNCTMKFGSWSYDKAKIDLVLIGSSMNLKDYWESGEWAIIKAPGYKHDIKYNCCEEIYPDITYSLYIRRLPLFYTINLIIPCLLISFLTVLVFYLPSDCGEKVTLCISVLLSLTVFLLVITETIPSTSLVIPLIGEYLLFTMIFVTLSIVITVFVLNVHYRTPTTHTMPSWVKTVFLNLLPRVMFMTRPTSNEGNAQKPRPLYGAELSNLNCFSRAESKGCKEGYPCQDGMCGYCHHRRIKISNFSANLTRSSSSESVDAVLSLSALSPEIKEAIQSVKYIAENMKAQNEAKEIQDDWKYVAMVIDRIFLWVFTLVCILGTAGLFLQPLMAREDA

SEQ ID NO: 39 - protein, 498 aa, Homo sapiens
MRRAPSLVLFFLVALCGRGNCRVANAEEKLMDDLLNKTRYNNLIRPATSSSQLISIKLQLSLAQLISVNEREQIMTTNVWLKQEWTDYRLTWNSSRYEGVNILRIPAKRIWLPDIVLYNNADGTYEVSVYTNLIVRSNGSVLWLPPAIYKSACKIEVKYFPFDQQNCTLKFRSWTYDHTEIDMVLMTPTASMDDFTPSGEWDIVALPGRRTVNPQDPSYVDVTYDFIIKRKPLFYTINLIIPCVLTTLLAILVFYLPSDCGEKMTLCISVLLALTFFLLLISKIVPPTSLDVPLIGKYLMFTMVLVTFSIVTSVCVLNVHHRSPSTHTMAPWVKRCFLHKLPTFLFMKRPGPDSSPARAFPPSKSCVTKPEATATSTSPSNFYGNSMYFVNPASAASKSPAGSTPVAIPRDFWLRSSGRFRQDVQEALEGVSFIAQHMKNDDEDQSVVEDWKYVAMVVDRLFLWVFMFVCVLGTVGLFLPPLFQTHAASEGPYAAQRD

SEQ ID NO: 40 - DNA, 31 bp, Homo sapiens
  variation (16)..(16): n= C or T
gctatggatgttcaanttgtgtgggaaatac

SEQ ID NO: 41 - DNA, 31 bp, Homo sapiens
  variation (16)..(16): n= A, G or T
tagaccaaagagccanttgcactccaggcta

SEQ ID NO: 42 - DNA, 31 bp, Homo sapiens
  variation (16)..(16): n= C or T
acctggggcaggttcnatcccttgcagttag

SEQ ID NO: 43 - DNA, 31 bp, Homo sapiens
  variation (16)..(16): n= A, G, C or T
cctcccttgccagtcngcctgtcttcctccc

SEQ ID NO: 44 - DNA, 31 bp, Homo sapiens
  variation (16)..(16): n= A or G
taattaagaaactaantctagcaggctaaag

SEQ ID NO: 45 - DNA, 31 bp, Homo sapiens
  variation (16)..(16): n= A or G
caatcattctgtccantccagccaaggttcc

SEQ ID NO: 46 - DNA, 31 bp, Homo sapiens
  variation (16)..(16): n= C or A
accgaataaccgctgntaatatggcccccgc

SEQ ID NO: 47 - DNA, 31 bp, Homo sapiens
  variation (16)..(16): n= C or T
ctttctgttctttttntttcttctcacgctc

SEQ ID NO: 48 - DNA, 31 bp, Homo sapiens
  variation (16)..(16): n=A or G
tatgactccaggaccnctatattttctccag

SEQ ID NO: 49 - DNA, 31 bp, Homo sapiens
  variation (16)..(16): n= A or G
gaagatatgactccangaccactatattttc

SEQ ID NO: 50 - DNA, 31 bp, Homo sapiens
  variation (16)..(16): n= A or G
atgttctgactaatgnactaaggctcattgc

SEQ ID NO: 51 - DNA, 31 bp, Homo sapiens
  variation (16)..(16): n= C or G
tgtttgggaattttancaaatgatggtgttt

SEQ ID NO: 52 - DNA, 31 bp, Homo sapiens
  variation (16)..(16): n= A or G
cttgcagtgtggcagncataacttctgatgc

SEQ ID NO: 53 - DNA, 31 bp, Homo sapiens
  variation (16)..(16): n= C or T
tgtggcaggcataacntctgatgctaatgtt

SEQ ID NO: 54 - DNA, 31 bp, Homo sapiens
  variation (16)..(16): n= C or A
agctattggacatgcnggcacctgtttggga

SEQ ID NO: 55 - DNA, 31 bp, Homo sapiens
  variation (16)..(16): n= A or G
tgtgtggaagattaanacgtccatgaagggt

SEQ ID NO: 56 - DNA, 31 bp, Homo sapiens
  variation (16)..(16): n= C or G
ttcgctacattacaanacacatcatgaagga

SEQ ID NO: 57 - DNA, 31 bp, Homo sapiens
  variation (16)..(16): n= C or T
gtcagacacgttggcnacaggccggatgatc

SEQ ID NO: 58 - DNA, 31 bp, Homo sapiens
  variation (16)..(16): n= C or T
aggctgcaaggagggntacccctgccaggac

SEQ ID NO: 59 - DNA, 31 bp, Homo sapiens
  variation (16)..(16): n=G or A
ttttgctcctaagggnacatttaacaatgga

SEQ ID NO: 60 - DNA, 31 bp, Homo sapiens
  variation (16)..(16): n= C or T
tcagccttagcactanggacattttgggctg

SEQ ID NO: 61 - DNA, 31 bp, Homo sapiens
  variation (16)..(16): n= G or A
tccacccagtttatgntgtactaagacagtt

SEQ ID NO: 62 - DNA, 31 bp, Homo sapiens
  variation (16)..(16): n= C or A
agcagcagcagcagcngccgcggcggcgaca

SEQ ID NO: 63 - DNA, 31 bp, Homo sapiens
  variation (16)..(16): n= C or G
agcagcagcagcagcngcggcggcgacagcg

SEQ ID NO: 64 - DNA, 31 bp, Homo sapiens
  variation (16)..(16): n= C or T
agctagaaggcacccnttgaggaagaggacc

(19) United States
(12) Patent Application Publication
(10) Pub. No.: US 2011/0039344 A1
(43) Pub. Date: Feb. 17, 2011

(54) METHOD AND TESTING A SUBJECT THOUGHT TO BE PREDISPOSED TO LUNG CANCER

(75) Inventors: Paul Brennan, Lyon (FR); Ivo Glynne Gut, Paris (FR); Simon Heath, Paris (FR); Mark Lathrop, Paris (FR)

Correspondence Address: FOLEY AND LARDNER LLP;SUITE 500, 3000 K STREET NW, WASHINGTON, DC, 20007, US

(73) Assignee: COMMISSARIAT A L'ENERGIE ATOMIQUE

(21) Appl. No.: 12/735,037

(22) PCT Filed: Dec. 12, 2008
(86) PCT No.: PCT/EP2008/067406

§ 371 Date: Jun. 11, 2010

(30) Foreign Application Priority Data
Dec. 12, 2007 (EP) 07301664.4

(51) Int. Cl. G01N 33/50

(52) U.S. Cl. 436/94

(57) ABSTRACT

The present invention concerns a method of testing a human thought to be predisposed to having lung cancer which comprises the step of analyzing a biological sample from said human for detecting the presence of a polymorphism on chromosome 15q25 associated with lung cancer.

This patent application claims the priority of European patent application EP 07301664.4 filed on Dec. 12, 2007, which is incorporated herein by reference.

### FIELD OF THE INVENTION

The present invention relates to lung cancer, and more precisely to a method of testing a subject thought to be predisposed to lung cancer.

### BACKGROUND OF THE INVENTION

Lung cancer is the most common cause of cancer death worldwide with over 1 million cases annually. This cancer is caused predominantly by tobacco smoking, with cessation of tobacco consumption being the primary method for prevention. The risk among those who quit smoking remains elevated (although less than those who continue to smoke), and former smokers make up an increasing proportion of lung cancer patients in countries where tobacco consumption has declined.

Actually treatment strategies are of limited efficacy, with an overall 5-year survival rate of about 15%.

Since lung cancer has an important heritable component, then identifying genes that are involved in its etiology may suggest chemoprevention targets or allow for identification of groups at high risk.

Nevertheless, and despite a large number of studies including both sporadic and multi-case families, success in identifying genes that cause lung cancer has been extremely limited.

As an example of such studies, one can cite the International patent application WO 2006/123955 disclosing the analysis of a large number of polymorphisms so as to establish a risk of cancer.

### SUMMARY OF THE INVENTION

The present invention relates to a method of testing a human thought to be predisposed to having lung cancer which comprises the step of i) analyzing a biological sample from said human for detecting the presence of a polymorphism on chromosome 15q25 associated with lung cancer.

In a preferred embodiment, said polymorphism on chromosome 15q25 associated with lung cancer corresponds to a single nucleotide polymorphism (SNP).

Preferably, said single nucleotide polymorphism is selected from the group comprising rs17483548 (nucleotide N at position 16 of SEQ ID NO:1, wherein allele A is associated to lung cancer), rs17405217 (nucleotide N at position 16 of SEQ ID NO:2, wherein allele T is associated to lung cancer), rs17483721 (nucleotide N at position 16 of SEQ ID NO:3, wherein allele C is associated to lung cancer), rs2656052 (nucleotide N at position 16 of SEQ ID NO:4, wherein allele G is associated to lung cancer), rs2568494 (nucleotide N at position 16 of SEQ ID NO:5, wherein allele A is associated to lung cancer), rs7181486 (nucleotide N at position 16 of SEQ ID NO:6, wherein allele C is associated to lung cancer), rs17483929 (nucleotide N at position 16 of SEQ ID NO:7, wherein allele A is associated to lung cancer), rs2656065 (nucleotide N at position 16 of SEQ ID NO:8, wherein allele T is associated to lung cancer), rs2009746 (nucleotide N at position 16 of SEQ ID NO:9, wherein allele C is associated to lung cancer), rs17484235 (nucleotide N at position 16 of SEQ ID NO:10, wherein allele G is associated to lung cancer), rs1504550 (nucleotide N at position 16 of SEQ ID NO:11, wherein allele C is associated to lung cancer), rs17484524 (nucleotide N at position 16 of SEQ ID NO:12, wherein allele G is associated to lung cancer), rs9788721 (nucleotide N at position 16 of SEQ ID NO:13, wherein allele C is associated to lung cancer), rs8034191 (nucleotide N at position 16 of SEQ ID NO:14, wherein allele C is associated to lung cancer), rs10519203 (nucleotide N at position 16 of SEQ ID NO:15, wherein allele G is associated to lung cancer), rs8031948 (nucleotide N at position 16 of SEQ ID NO:16, wherein allele T is associated to lung cancer), rs931794 (nucleotide N at position 31 of SEQ ID NO:17, wherein allele G is associated to lung cancer), rs2036527 (nucleotide N at position 31 of SEQ ID NO:18, wherein allele A is associated to lung cancer), rs17486278 (nucleotide N at position 16 of SEQ ID NO:19, wherein allele C is associated to lung cancer), rs7180002 (nucleotide N at position 16 of SEQ ID NO:20, wherein allele T is associated to lung cancer), rs951266 (nucleotide N at position 16 of SEQ ID NO:21, wherein allele T is associated to lung cancer), rs16969968 (nucleotide N at position 16 of SEQ ID NO:22, wherein allele A is associated to lung cancer), rs1051730 (nucleotide N at position 31 of SEQ ID NO:23, wherein allele A is associated to lung cancer), rs1317286 (nucleotide N at position 16 of SEQ ID NO:24, wherein allele G is associated to lung cancer), and rs17487223 (nucleotide N at position 16 of SEQ ID NO:25, wherein allele T is associated to lung cancer).

In a preferred embodiment, the method of the invention further comprises the step of ii) determining the cumulative risks for said human to be predisposed to having a lung cancer by further determining the smoking pattern of said human.

The present invention also relates to kits that comprise, e.g., probes for identifying the polymorphism herein, e.g., packaged in suitable containers with instructions for correlating detected polymorphism to lung cancer predisposition.

Finally, the present invention also relates to a method of identifying modulators of lung cancer comprising the step of contacting a potential modulator to a protein selected in the group comprising IREB2, such as SEQ ID NO: 33, LOC123688, such as SEQ ID NO: 34 and SEQ ID NO:35, PSMA4, such as SEQ ID NO: 36, CHRNA5, such as SEQ ID NO: 37, CHRNA3, such as SEQ ID NO: 38, and CHRNB4, such as SEQ ID NO: 39, or to a nucleic acid that encodes such a protein. An effect of the potential modulator on the gene or gene product is detected, thereby identifying whether the potential modulator modulates lung cancer.

### BRIEF DESCRIPTION OF THE DRAWINGS

FIG. 1 shows (a) the QQ plot for bottom 90% of p-value and (b) top 10% of p-value, as well as (c) scatter plot of p-value in −log scale from the trend test for 310,023 genotyped variants comparing 1,926 lung cancer cases and 2,522 controls.

FIG. 2 shows the lung cancer area of interest across 15q25.

FIG. 3 shows the Odds Ratio (OR) and 95% confidence interval (CI) for lung cancer comparing heterozygous (T/C) and homozygous (C/C) genotypes of rs8034191 to homozygous (T/T) genotype.

FIG. 4 shows the relevance of smoking and of rs8034191 genotype to cancer mortality depending of the subject age.

FIG. 5 shows the OR and 95% CI for upper aero-digestive for the rs8034191 variant.

### DETAILED DESCRIPTION

The present invention is based on the discovery by the present inventors that polymorphisms located on chromosome 15q25 are correlated with lung cancer predisposition.

It was already known from the prior art that some genes located on chromosome 15q25 were associated with nicotine dependence (SACCONE et al., Hum. Mol. Genet., vol. 16, p: 36-49, 2007). Moreover, the smokers having an important nicotine dependence have most of the time a higher tar exposure (i.e., resulting from a higher smoking consumption) and thus a higher risk of developing a lung cancer (See conclusion of KUNZE et al., Atemw.-Lungenkrkh., vol. 4(1), S95-96, 1998). Nevertheless, the method of testing a human thought to be predisposed to lung cancer of the prior art used directly the step of determining the smoking pattern (i.e., the tar exposure) of said human instead of nicotine dependence parameters. In fact, the markers of nicotine dependence were not considered as real lung cancer markers since they should include a bias in determining a predisposition to lung cancer compared to the more direct tar exposure parameter.

Presently, the inventors have also established that the risks of developing a lung cancer associated with polymorphisms located on chromosome 15q25 are cumulative with the risks associated with the smoking pattern of a given human. Consequently, said polymorphisms correspond to direct lung cancer predisposition markers such as the smoking pattern parameter, and are independent, at least partly, of said smoking pattern parameter.

Thus, in a first aspect, the present invention provides a method of testing a human thought to be predisposed to having lung cancer which comprises the step of i) analyzing a biological sample from said human for detecting the presence of a polymorphism on chromosome 15q25 associated with lung cancer.

Said method, by detecting the presence of a polymorphism on chromosome 15q25 associated with lung cancer in a human enables to confirm that said human has or is predisposed for having lung cancer.

Preferably, a polymorphism on chromosome 15q25 corresponds to a polymorphism located between position 76,480,000 and 76,800,000 of the human chromosome 15 (NC_{—}000015), preferably between position 76,499,754 and 76,795,005, and most preferably between position 76,517,000 and 76,711,000.

As used herein, the expression “biological sample” refers to solid tissues such as, for example, a lung biopsy; buccal swab, fluids and excretions such as for example, sputum, induced sputum, blood, serum, plasma, urine. Preferably, said biological sample is a fluid sample and most preferably a blood sample. Again preferably, said human is a Caucasian.

Typically, the polymorphism on chromosome 15q25 associated with lung cancer corresponds to a single nucleotide polymorphism (SNP), which corresponds to a mutation of a single base pair, which mutation can exist in the human population with a frequency inferior to 1%, to 0.1%, to 0.01%, and even inferior to 0.001%.

Preferably, said single nucleotide polymorphism is selected from the group comprising rs17483548 (nucleotide N at position 16 of SEQ ID NO:1, wherein allele A is associated to lung cancer), rs17405217 (nucleotide N at position 16 of SEQ ID NO:2, wherein allele T is associated to lung cancer), rs17483721 (nucleotide N at position 16 of SEQ ID NO:3, wherein allele C is associated to lung cancer), rs2656052 (nucleotide N at position 16 of SEQ ID NO:4, wherein allele G is associated to lung cancer), rs2568494 (nucleotide N at position 16 of SEQ ID NO:5, wherein allele A is associated to lung cancer), rs7181486 (nucleotide N at position 16 of SEQ ID NO:6, wherein allele C is associated to lung cancer), rs17483929 (nucleotide N at position 16 of SEQ ID NO:7, wherein allele A is associated to lung cancer), rs2656065 (nucleotide N at position 16 of SEQ ID NO:8, wherein allele T is associated to lung cancer), rs2009746 (nucleotide N at position 16 of SEQ ID NO:9, wherein allele C is associated to lung cancer), rs17484235 (nucleotide N at position 16 of SEQ ID NO:10, wherein allele G is associated to lung cancer), rs1504550 (nucleotide N at position 16 of SEQ ID NO:11, wherein allele C is associated to lung cancer), rs17484524 (nucleotide N at position 16 of SEQ ID NO:12, wherein allele G is associated to lung cancer), rs9788721 (nucleotide N at position 16 of SEQ ID NO:13, wherein allele C is associated to lung cancer), rs8034191 (nucleotide N at position 16 of SEQ ID NO:14, wherein allele C is associated to lung cancer), rs10519203 (nucleotide N at position 16 of SEQ ID NO:15, wherein allele G is associated to lung cancer), rs8031948 (nucleotide N at position 16 of SEQ ID NO:16, wherein allele T is associated to lung cancer), rs931794 (nucleotide N at position 31 of SEQ ID NO:17, wherein allele G is associated to lung cancer), rs2036527 (nucleotide N at position 31 of SEQ ID NO:18, wherein allele A is associated to lung cancer), rs17486278 (nucleotide N at position 16 of SEQ ID NO:19, wherein allele C is associated to lung cancer), rs7180002 (nucleotide N at position 16 of SEQ ID NO:20, wherein allele T is associated to lung cancer), rs951266 (nucleotide N at position 16 of SEQ ID NO:21, wherein allele T is associated to lung cancer), rs16969968 (nucleotide N at position 16 of SEQ ID NO:22, wherein allele A is associated to lung cancer), rs1051730 (nucleotide N at position 31 of SEQ ID NO:23, wherein allele A is associated to lung cancer), rs1317286 (nucleotide N at position 16 of SEQ ID NO:24, wherein allele G is associated to lung cancer), and rs17487223 (nucleotide N at position 16 of SEQ ID NO:25, wherein allele T is associated to lung cancer).

The single nucleotide polymorphism on chromosome 15q25 associated with lung cancer may also include a single nucleotide polymorphism selected in the group comprising rs7177092 (nucleotide N at position 16 of SEQ ID NO:40), rs8032410 (nucleotide N at position 16 of SEQ ID NO:41), rs2055588 (nucleotide N at position 16 of SEQ ID NO:42), rs954144 (nucleotide N at position 16 of SEQ ID NO:43), rs8033501(nucleotide N at position 16 of SEQ ID NO:44), rs3743080 (nucleotide N at position 16 of SEQ ID NO:45), rs3813571 (nucleotide N at position 16 of SEQ ID NO:46), rs1065640 (nucleotide N at position 16 of SEQ ID NO:47), rs11551787 (nucleotide N at position 16 of SEQ ID NO:48), rs11551783 (nucleotide N at position 16 of SEQ ID NO:49), rs11551782 (nucleotide N at position 16 of SEQ ID NO:50), rs11551779 (nucleotide N at position 16 of SEQ ID NO:51), rs11551781 (nucleotide N at position 16 of SEQ ID NO:52), rs11551786 (nucleotide N at position 16 of SEQ ID NO:53), rs11551784 (nucleotide N at position 16 of SEQ ID NO:54), rs1052040 (nucleotide N at position 16 of SEQ ID NO:55), rs1042500 (nucleotide N at position 16 of SEQ ID NO:56), rs8040868 (nucleotide N at position 16 of SEQ ID NO:57), rs1051731 (nucleotide N at position 16 of SEQ ID NO:58), rs660652 (nucleotide N at position 16 of SEQ ID NO:59), rs472054 (nucleotide N at position 16 of SEQ ID NO:60), rs8029939 (nucleotide N at position 16 of SEQ ID NO:61), rs12906406 (nucleotide N at position 16 of SEQ ID NO:62), rs12906525 (nucleotide N at position 16 of SEQ ID NO:63), and rs12904278 (nucleotide N at position 16 of SEQ ID NO:64).

Most preferably, said polymorphism is a single nucleotide polymorphism on chromosome 15q25 associated with lung cancer, wherein said single nucleotide polymorphism is selected from the group comprising rs2656052 (nucleotide N at position 16 of SEQ ID NO:4, wherein allele G is associated to lung cancer), rs17484235 (nucleotide N at position 16 of SEQ ID NO:10, wherein allele G is associated to lung cancer), rs8034191 (nucleotide N at position 16 of SEQ ID NO:14, wherein allele C is associated to lung cancer), rs10519203 (nucleotide N at position 16 of SEQ ID NO:15, wherein allele G is associated to lung cancer), rs8031948 (nucleotide N at position 16 of SEQ ID NO:16, wherein allele T is associated to lung cancer), rs931794 (nucleotide N at position 31 of SEQ ID NO:17, wherein allele G is associated to lung cancer), rs2036527 (nucleotide N at position 31 of SEQ ID NO:18, wherein allele A is associated to lung cancer), rs16969968 (nucleotide N at position 16 of SEQ ID NO:22, wherein allele A is associated to lung cancer), and rs1317286 (nucleotide N at position 16 of SEQ ID NO:24, wherein allele G is associated to lung cancer.

Typical techniques for detecting a polymorphism may include restriction fragment length polymorphism, hybridization techniques, DNA sequencing, exonuclease resistance, microsequencing, solid phase extension using ddNTPs, extension in solution using ddNTPs, oligonucleotide ligation assays, methods for detecting single nucleotide polymorphisms such as dynamic allele-specific hybridization, ligation chain reaction, mini-sequencing, DNA “chips”, allele-specific oligonucleotide hybridization with single or dual-labeled probes merged with PCR or with molecular beacons, and others.

Preferably, said technique for detecting a polymorphism is selected in the group comprising methods for detecting single nucleotide polymorphisms.

Depending on the polymorphism, the technique used for its detection can also be based on the analysis of mRNA transcript from a gene located on chromosome 15q25, if said polymorphism is also present in said mRNA sequence. As an example, said mRNA transcript is selected in the group comprising IREB2 mRNA, such as SEQ ID NO: 26, LOC123688 mRNA, such as SEQ ID NO: 27 and SEQ ID NO: 28, PSMA4 mRNA, such as SEQ ID NO: 29, CHRNA5 mRNA, such as SEQ ID NO: 30, CHRNA3 mRNA, such as SEQ ID NO: 31, and CHRNB4 mRNA, such as SEQ ID NO: 32. As an example of a single nucleotide polymorphism on chromosome 15q25 associated with lung cancer which is also present in the mRNA sequence, one can cite rs16969968 and rs1051730. This polymorphism analysis can be assessed by preparing mRNA/cDNA from cells in a biological sample from a human, and hybridizing the mRNA/cDNA with a reference polynucleotide. The prepared mRNA/cDNA can be used in hybridization or amplification assays that include, but are not limited to, polymerase chain reaction analyses, such as quantitative PCR (TaqMan), and probes arrays such as GeneChip™ DNA Arrays (AFFYMETRIX).

Still depending on the polymorphism, the technique used for its detection can also be based on the analysis of the protein translated from a gene located on chromosome 15q25, if said polymorphism is also present in said protein sequence. As an example, said protein is selected in the group comprising IREB2, such as SEQ ID NO: 33, LOC123688, such as SEQ ID NO: 34 and SEQ ID NO:35, PSMA4, such as SEQ ID NO: 36, CHRNA5, such as SEQ ID NO: 37, CHRNA3, such as SEQ ID NO: 38, and CHRNB4, such as SEQ ID NO: 39. As an example of a single nucleotide polymorphism on chromosome 15q25 associated with lung cancer which is also present in the protein sequence, one can cite rs16969968 (substitution of aspartic acid (D) to asparagine (B) at amino acid position 398 (D398N) of the CHRNA5 protein). This polymorphism analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin), or an antibody fragment (e.g., a single-chain antibody, an isolated antibody hypervariable domain, etc.) which binds or does not bind specifically to the protein comprising the analyzed polymorphism.

Said analysis can be assessed by a variety of techniques well known by one of skill in the art including, but not limited to, enzyme immunoassay (EIA), radioimmunoassay (RIA), Western blot analysis and enzyme linked immunoabsorbant assay (ELISA).

Polyclonal antibodies can be prepared by immunizing a suitable animal, such as mouse, rabbit or goat, with a protein comprising the analyzed polymorphism, translated from a gene located on chromosome 15q25, or a fragment thereof. The antibody titer in the immunized animal can be monitored over time by standard techniques, such as with an ELISA using immobilized polypeptide. At an appropriate time after immunization, e.g., when the specific antibody titers are highest, antibody producing cells can be obtained from the animal and used to prepare monoclonal antibodies (mAb) by standard techniques, such as the hybridoma technique originally described by KOHLER and MILSTEIN (Nature, vol. 256, p: 495-497, 1975), the human B cell hybridoma technique (KOZBOR et al., Immunol., vol. 4, p: 72, 1983), the EBV-hybridoma technique (COLE et al., In Monoclonal Antibodies and Cancer Therapy, Alan R. Liss, Inc., p: 77-96, 1985) or trioma techniques. The technology for producing hybridomas is well known (see generally Current Protocols in Immunology, COLIGAN et al. ed., John Wiley & Sons, New York, 1994). Hybridoma cells producing the desired monoclonal antibody are detected by screening the hybridoma culture supernatants for antibodies that bind the polypeptide of interest, e.g., using a standard ELISA.

In a preferred embodiment, the method of the invention further comprises the step of ii) determining the cumulative risks for said human to be predisposed to having a lung cancer by further determining the smoking pattern of said human.

In fact, and as established by the inventors, the relative risks associated with the “lung cancer” haplotypes are cumulative with the relative risks associated with various smoking pattern (See Example, part 5).

In a second aspect, the present invention concerns a kit that comprise, e.g., probes for identifying the polymorphism herein, e.g., packaged in suitable containers with instructions for correlating detected polymorphism to lung cancer predisposition.

In a third aspect, the present invention concerns a method of identifying modulators of lung cancer comprising the step of contacting a potential modulator to a protein selected in the group comprising IREB2, such as SEQ ID NO: 33, LOC123688, such as SEQ ID NO: 34 and SEQ ID NO:35, PSMA4, such as SEQ ID NO: 36, CHRNA5, such as SEQ ID NO: 37, CHRNA3, such as SEQ ID NO: 38, and CHRNB4, such as SEQ ID NO: 39, or to a nucleic acid that encodes such a protein. An effect of the potential modulator on the gene or gene product is detected, thereby identifying whether the potential modulator modulates lung cancer.

The gene or gene product that is contacted by the modulator can include any allelic form known for any of the IREB2, LOC123688, PSMA4, CHRNA5, CHRNA3 or the CHRNB4 gene. Any allelic forms, whether genes, RNAs or proteins, that positively correlate to lung cancer are preferred targets for modulator screening.

Effects of interest that can be screened for include: (a) increased or decreased expression of any of the IREB2, LOC123688, PSMA4, CHRNA5, CHRNA3 or the CHRNB4 gene, and/or any protein encoded by these genes, in the presence of a modulator; (b) a change in the timing or location of expression of any of the IREB2, LOC123688, PSMA4, CHRNA5, CHRNA3 or the CHRNB4 gene, and/or any protein encoded by these genes, in the presence of the modulator; (c) a change in any activity (e.g. increased or decreased activity) of the gene product of any of the IREB2, LOC123688, PSMA4, CHRNA5, CHRNA3 or the CHRNB4 gene in the presence of a modulator; and/or (d) a change in localization of proteins encoded by any of the IREB2, LOC123688, PSMA4, CHRNA5, CHRNA3 or the CHRNB4 gene in the presence of the modulator.

The precise format of the modulator screen will, of course, vary, depending on the effect(s) being detected and the equipment available. Northern analysis, quantitative RT-PCR and/or array-based detection formats can be used to distinguish expression levels or patterns of genes noted above. Protein expression levels can also be detected using available methods, such as western blotting, ELISA analysis, antibody hybridization, BIAcore, or the like. Any of these methods can be used to distinguish changes in expression levels of any of the IREB2, LOC123688, PSMA4, CHRNA5, CHRNA3 or the CHRNB4 gene, or the RNA or proteins encoded therein that result from a potential modulator.

Accordingly, one may screen for potential modulators of any of the IREB2, LOC123688, PSMA4, CHRNA5, CHRNA3 or the CHRNB4 gene and/or the RNA and protein encoded therein for activity or expression. For example, potential modulators (small molecules, RNAs (e.g., RNAi), organic molecules, inorganic molecules, proteins, hormones, transcription factors, or the like) can be contacted to a cell comprising an allele of interest and an effect on activity or expression (or both) of a gene, RNA or protein corresponding to any of the IREB2, LOC123688, PSMA4, CHRNA5, CHRNA3 or the CHRNB4 gene. For example, expression of any of the IREB2, LOC123688, PSMA4, CHRNA5, CHRNA3 or the CHRNB4 gene can be detected, e.g., via Northern analysis or quantitative (optionally real time) RT-PCR, before and after application of potential expression modulators. Similarly, promoter regions of the various genes (e.g., generally sequences in the region of the start site of transcription, e.g., within 5 kb of the start site, e.g., 1 kb, or less e.g., within 500 bp or 250 bp or 100 bp of the start site) can be coupled to reporter constructs (CAT, beta-galactosidase, luciferase or any other available reporter) and can be similarly tested for expression activity modulation by the potential modulator. In either case, the assays can be performed in a high-throughput fashion, e.g., using automated fluid handling and/or detection systems, in serial or parallel fashion. Similarly, activity modulators can be tested by contacting a potential modulator to an appropriate cell using any of the activity detection methods herein, regardless of whether the activity that is detected is the result of activity modulation, expression modulation or both. These assays can be in vitro, cell-based, or can be screens for modulator activity performed on laboratory animals such as knock-out transgenic mice comprising a gene of interest.

Whole animal assays can also be used to assess the effects of modulators on cells or whole animals (e.g., transgenic knock-out mice), e.g., by monitoring an effect on a cell-based phenomenon, a change in displayed animal phenotype, or the like.

Potential modulator libraries to be screened for effects on expression and/or activity are available. These libraries can be random, or can be targeted. For example, a modulator library may be screened for effects on expression of any of the IREB2, LOC123688, PSMA4, CHRNA5, CHRNA3 or the CHRNB4 gene.

Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of any of the IREB2, LOC123688, PSMA4, CHRNA5, CHRNA3 or the CHRNB4 gene or gene products is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones. Commercially available libraries and library design services include those offered by CHEMICAL DIVERSITY (San Diego, Calif.), AFFYMETRIX (Santa Clara, Calif.), SIGMA (St. Louis Mo.), CHEMBRIDGE RESEARCH LABORATORIES (San Diego, Calif.), TIMTEC (Newark, Del.), NUEVOLUTION A/S (Copenhagen, Denmark) and many others.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLES

### 1) Genome Wide Analysis

We conducted a genome-wide association study of lung cancer using the ILLUMINA SENTRIX® HumanHap300 BeadChip that contains 317,139 SNPs and is estimated to tag approximately 80% of common genomic variation (BARRETT & CARDON, Nat. Genet., vol. 38, p: 659-662, 2006).

We initially genotyped 1989 cases and 2625 controls from the IARC central Europe lung cancer study.

The IARC study of lung cancer in central Europe was conducted with cancer institutes in 6 countries including Czech Republic (Prague, Olomouc, Brno), Hungary (Borsod, Heves, Szabolcs, Szolnok, Budapest), Poland (Warsaw, Lodz), Romania (Bucharest), Russia (Moscow) and Slovakia (Banska Bystrica, Bratislava, Nitra) between 1999 and 2002.

Briefly, each centre followed an identical protocol and was responsible for recruiting a consecutive group of newly diagnosed cases of lung cancer and a comparable group of hospital or population controls. All subjects were interviewed based on a standard questionnaire and information on lifestyle risk factors, occupational history; medical and family history has been collected. Written consent for participation was obtained from all study subjects and ethical approval has been obtained for all study centers as well as at IARC, the coordinating center. Controls in all centers except Warsaw were chosen among subjects admitted as in-patients or out-patients in the same hospital as the cases with conditions unrelated to tobacco including minor surgical conditions, benign disorders, common infections, eye conditions (except cataract or diabetic retinopathy) and common orthopeadic diseases (except osteoporosis). In Warsaw, population controls were selected by random sampling from the Polish Electronic List of Residents. This resulted in a total of 3052 potential controls. Cases and controls were frequency matched by sex, age (+/−3 years), center, referral (or of residence) area and period of recruitment (+/−6 months). The participation rates for both cases and controls were over 80% in all centers. Blood samples were collected from all subjects and DNA has been extracted. Candidate gene studies based on TAQMAN genotyping have been completed on over 100 candidate gene variants (BRENNAN et al., Hum. Mol. Genet., vol. 16, 2007). 1989 lung cancer cases (434 adenocarcinoma, 815 squamous cell, 298 small cell and 379 of mixed cell or other histology) and 2625 controls had sufficient DNA amount and quality and was genotyped at Centre National Genotypage (CNG) with HUMANHAP300K BEADCHIP using the ILLUMINA INFINIUM PLATFORM, which included 317,139 SNPs (http://www.illumina.com/downloads/HUMAN HAP300Datasheet.pdf).

A systematic quality control was conducted on the raw ILLUMINA HUMANHAP300K genotyping data. This quality control has resulted in the exclusion of:

a) 6,107 variants with a genotype call rate of less than 95% were excluded, such as 50 individuals where the overall genotype completion rate was less than 90%.

b) 972 variants as their genotype distributions clearly deviated from that expected by HARDY-WEINBERG Equilibrium (HWE) among controls (p-value of less than 10⁻⁷).

c) 37 monomorphic variants.

d) 42 individuals as they showed sex discrepancies based on the heterozygosity rate from chromosome X.

e) 17 unexpected duplicates and 23 unexpected first degree relatives based on similarity of genotype.

f) 29 individuals where the HumanHap300K Illumina genotyping and Taqman genotyping from a previous candidate gene study (BRENNAN et al., abovementioned, 2007) of 40 overlapping variants showed >5% discordance.

The population outliers were detected using a set of 8,155 markers with low linkage disequilibrium (R²<0.001) to classify the subjects by Structure Association (FALUSH et al., Genetics, vol. 164 (4), p: 1567-87, 2003). The data from the founders of the HapMap trios of YRI (N=60), CEU (n=60) and Asian (CHB/JPT combined); n=90) were used as the internal controls for this analysis assuming admixture model (individuals can have a mixed ancestry). 5 outliers were identified and excluded from the analysis. A comparison of 310,023 SNPs between 1926 cases and 2522 controls were used in the final analysis.

### 2) Genome Wide Statistical Analysis

Each of the 310,023 SNPs was analyzed individually comparing common homozygotes to heterozygous and homozygous carriers of the variant allele by calculating p-values for trend (p_{trend}) in a logistic regression model and incorporating additional parameters including, country, age and sex.

For this analysis, the association between the 310,023 variants and the disease risk was estimated by the OR and 95% CI using the multivariate unconditional logistic regression using PLINK (PURCELL et al., Am J Hum. Genet., vol. 81, p: 559-575, 2007) assuming a co-dominant genetic model (the effect of the variant by log-additive model with 1 degree of freedom). Study matching variables of age, sex, and country of recruitment were included in the regression as covariates. The results that obtained a level of significance of p<5×10⁻⁷ were considered significant at a genome wide level.

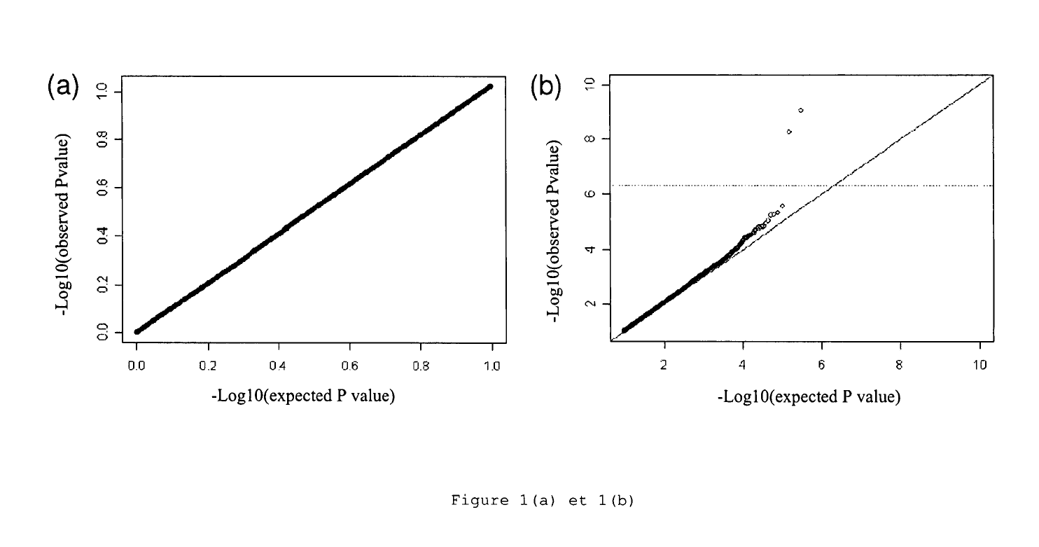

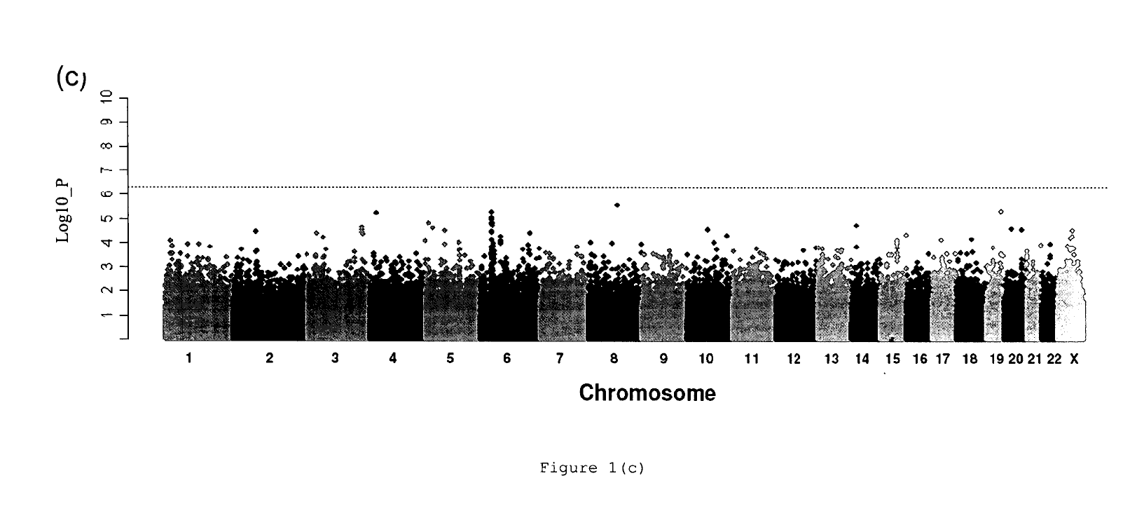

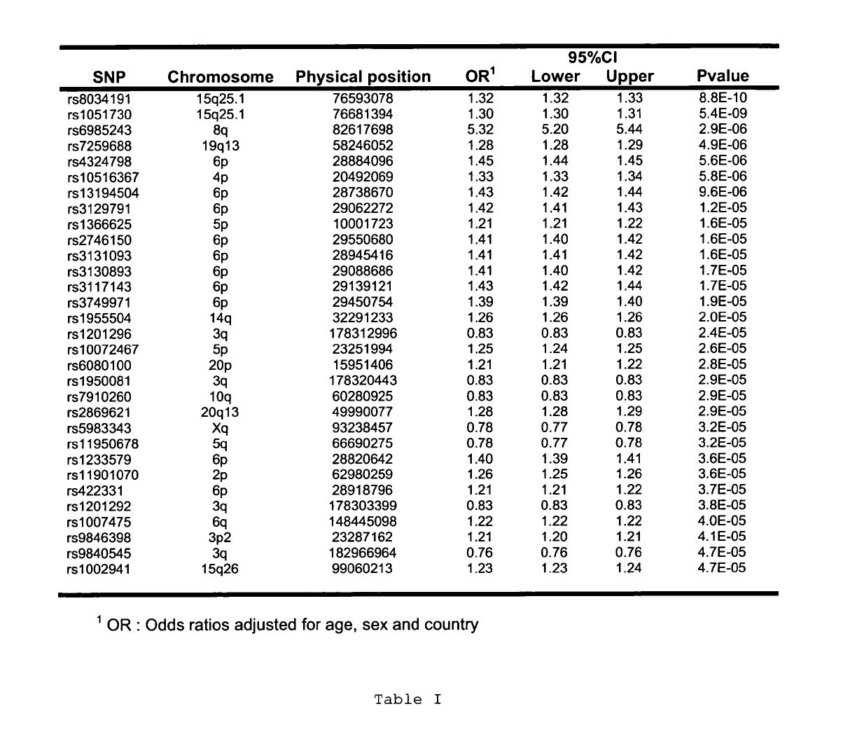

The FIG. 1 shows the (a) QQ plot for bottom 90% of p-values and (b) the top 10% of p-values, as well as (c) the scatter plot of p-values in −log scale from the trend test for the 310,023 genotyped variants comparing 1,926 lung cancer cases and 2,522 controls.

The results show that the distribution of the bottom 90% of p-values were similar to the expected distribution and the genomic control parameter was 1.03 implying that there was no systematic increase in false positive findings due to population stratification or any other form of bias (FIG. 1a). However, the results established that there was a strong deviation between the observed and expected p-values among the top 10% (FIG. 1b). In particular, two SNPs on chromosome 15q25, rs1051730 and rs8034191, were strongly associated with disease (p=5×10⁻⁹ and p=9×10⁻¹° respectively) exceeding the genome-wide significance level of 5×10⁻⁷ (FIG. 1c).

The principal components analysis implemented in the computer program EIGENSTRAT was used to control for potential effects of population stratification (PRICE et al., Nat. Genet., vol. 38, p: 904-909, 2006). Adjustment of the logistic regression with principal components marginally decreased the significance of the disease associations for these markers (p=7×10⁻⁹ and p=1×10⁻⁹, respectively). Additional analyses were undertaken with potential outliers eliminated based on the principal components analysis, and again did not significantly modify the conclusions.

The Table I identified the markers with p_{trend}<5×10⁻⁵ in the genome-wide association study.

The results have indicated that population stratification was unlikely to account for the observation of the strong association with the 15q25 markers, and no other region contained markers exceeding the genome-wide significance level. However, the results show that several regions contained SNPs with significance that exceeded 5×10⁻⁵ (see Table I).

### 3) Association of 15q25 Markers with Lung Cancer in IARC Cohort

The association was confirmed by genotyping 34 additional 15q25 markers that were selected as follows.

First, the imputation methods proposed by ABECASIS and colleagues (http://www.sph.umich.edu/csg/abecasis/MACH/index.html) was used to identify additional genetic variants from the CEU Hapmap data that are likely to have a strong disease association, but that are not present in the HumanHap 300 panel. It was attempted to genotype the SNPs from the 15q25 region for which the p-value of the association statistic obtained from the imputed data was <10⁻⁶.

Second, SNPs of CHRNA5 and CHRNA3 that had been included in a previous study of these genes in nicotine dependence (SACCONE et al., Hum. Mol. Genet., vol. 16, p: 36-49, 2007) were included.

Third, it was also attempted to genotype all non-synonymous SNPs in dbSNP from the six genes within or near the association region.

Follow up genotyping for the study was performed using the 5′ exonuclease assay (TAQMAN, APPLIED BIOSYSTEMS) at the IARC and TAQMAN and AMPLIFLUOR at CNG. Cases and controls were randomly mixed when genotyped and laboratory personnel were blinded to case/control status. A randomly selected 7% of the study subjects (both cases and controls) and re-genotyped for each polymorphism to examine the reliability of the genotyping assays. Internal duplicate concordance was >99.9% and genotyping success rate 97% or greater. Genotype distributions did not depart from HARDY-WEINBERG Equilibrium (HWE) in the controls. The concordance between the ILLUMINA and TAQMAN genotyping for rs8034191 was greater than 99.8% in the IARC central Europe study.

The replication analysis was conducted using SAS 9.1 software. The association between the variants and the disease risk was estimated by the OR and 95% CI using the multivariate unconditional logistic regression estimated the effect of the variant by co-dominant and genotype-specific models. Stratification has been done by smoking status and histological subtypes to evaluate the effect modification by smoking status and the effect heterogeneity across histological subtypes. To evaluate whether there are any subtle differences among central Europeans, the principal component analysis of the ILLUMINA HUMANHAP300K genotyping data was conducted and included the eigen values of the first three axes. This analysis was conduced using EIGENSTRAT (PRICE et al., above-mentioned, 2006). Briefly, this analysis consisted of two steps: (i) conducting principal components analysis to reduce the data into 3 axes, which refer to population subgroups (or ancestry); (ii) computing the association statistics adjusted for the inferred ancestry by including the eigen value into the logistic regression. As the results were almost identical with and without eigen values, it has been chosen to report the results without such eigen values adjustment.

The results for all markers tested in the 15q25 region, including those in the HumanHap 300 panel, are shown in Table II. In this table, the closest gene is indicated for markers within or near a gene. HapMap frequencies are provided when available. Finally, ORs and 95% CIs were calculated for polymorphic markers.

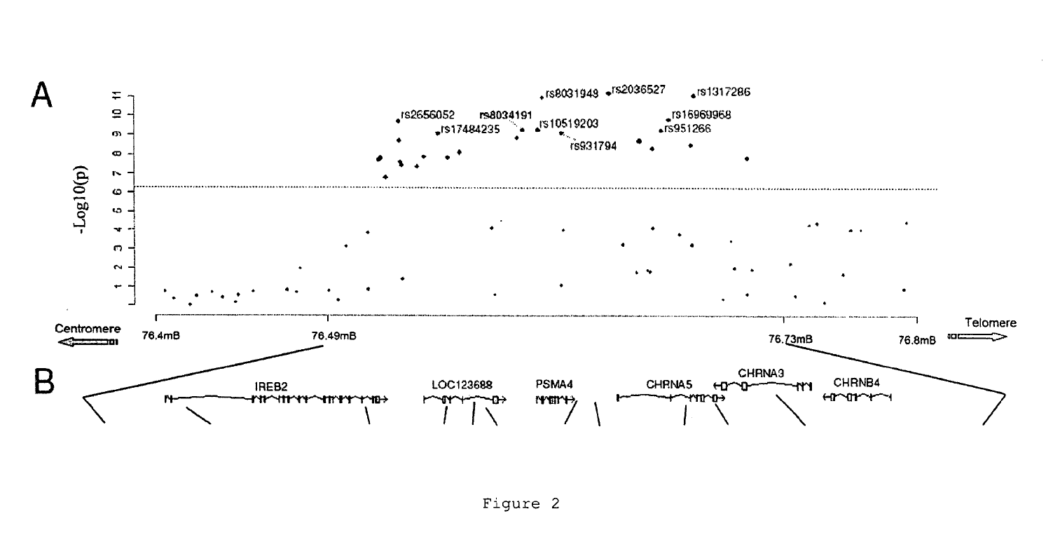

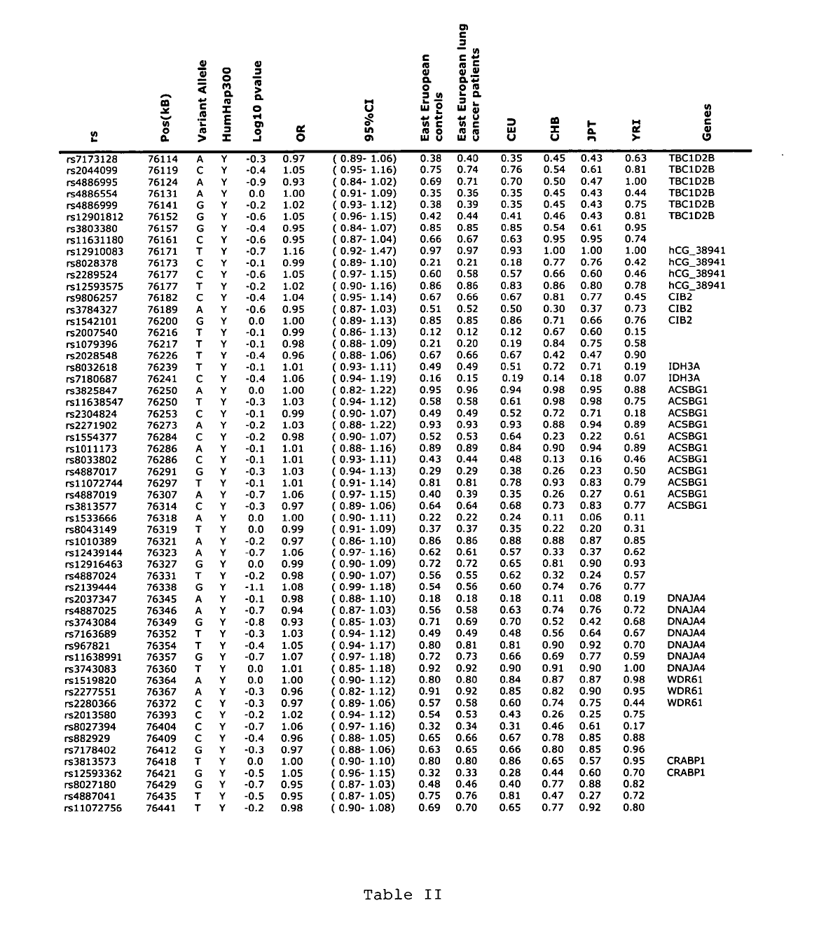

The FIG. 2 shows the lung cancer area of interest across 15q25. In this figure, the panel A shows p-values for SNPs genotyped in the 15q25 region (76.4-76.8 Mb). The dotted line indicates the threshold of p<5×10⁻⁷ at which results were considered genome-wide significant. Points labeled with “rs” numbers have a p<1×10⁻⁹. The panels B indicates the positions of the 6 known genes in the high LD genomic region approximately delineated by rs4887053 (76.49 Mb) and rs12594247 (76.73 Mb), which contains the SNPs strongly associated with lung cancer risk.

The Table III show the five common haplotypes (frequency >1%) across the 24 15q25 SNPps which achieved genome wide significance in the central Europe IARC study. The haplotype frequencies are shown combined and individually for each participating country. The alleles associated with increased risk (OR>1) are indicated in bold. It has to be noticed that the two predominant haplotypes account for 81% of the haplotypes in the central Europe IARC study and that three rare recombinant haplotypes are also present at frequencies >1%.

The results established that twenty five of the additional genotyped markers showed evidence of association exceeding the genome-wide significance level of 5×10⁻⁷ (see FIG. 2). These markers span more than 182 kb but are in strong linkage disequilibrium (pairwise D′>0.8 and r2>0.6) with two predominant haplotypes accounting for more than 89% of the haplotypes in patients and controls (see Table III).

### 4) Association of 15q25 Markers with Lung Cancer in Other Cohorts

In order to confirm the previous association, one of the principal disease associated SNPs (rs8034191) has been genotyped in 5 further independent studies of lung cancer. These were the EPIC cohort study (781 cases and 1578 controls), the CARET cohort study (764 cases and 1515 controls), the HUNT and Tromsø cohort studies (235 cases and 392 controls), the Liverpool lung cancer case-control study (408 cases and 814 controls), and the Toronto lung cancer case-control study (330 cases and 453 controls).

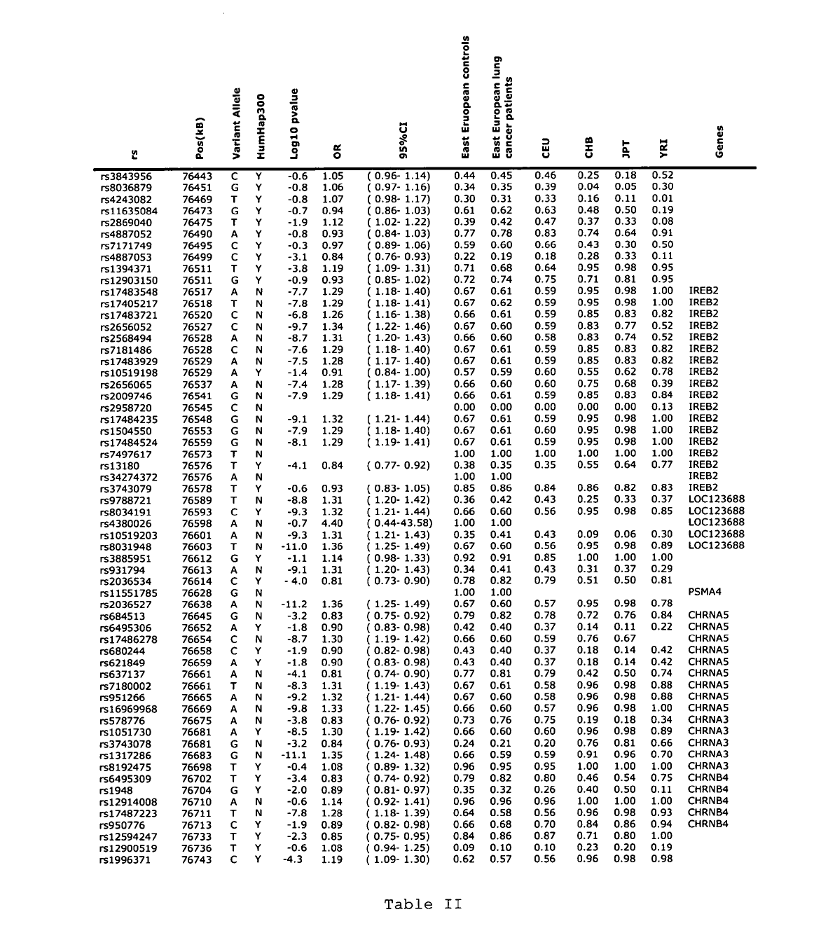

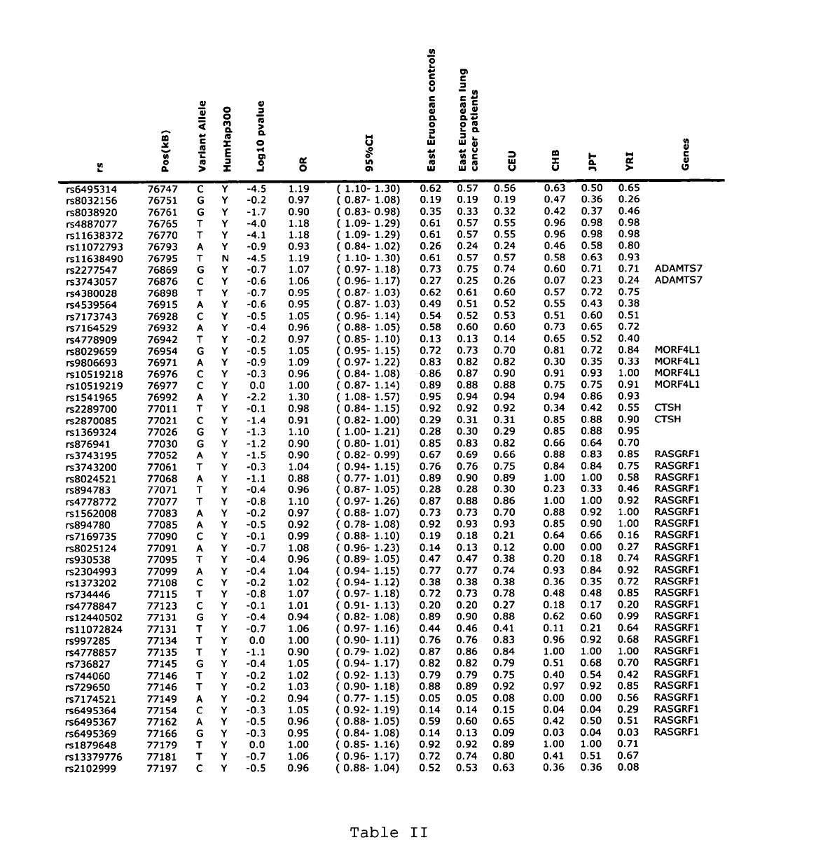

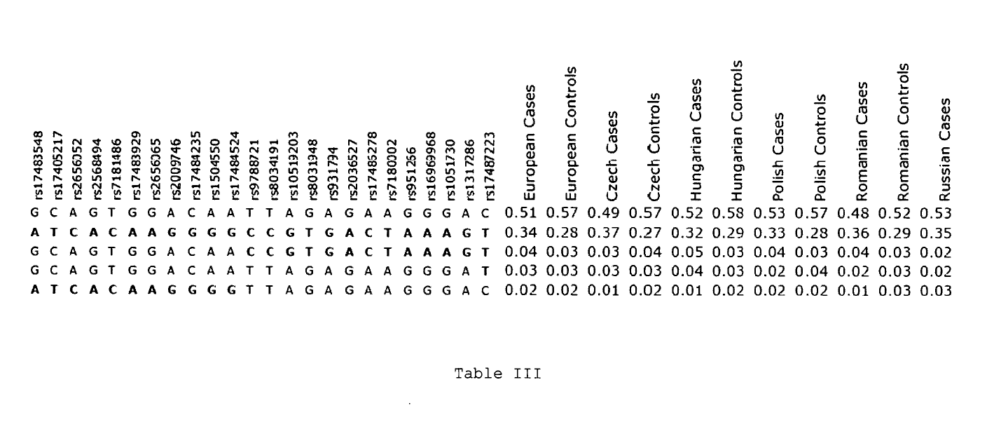

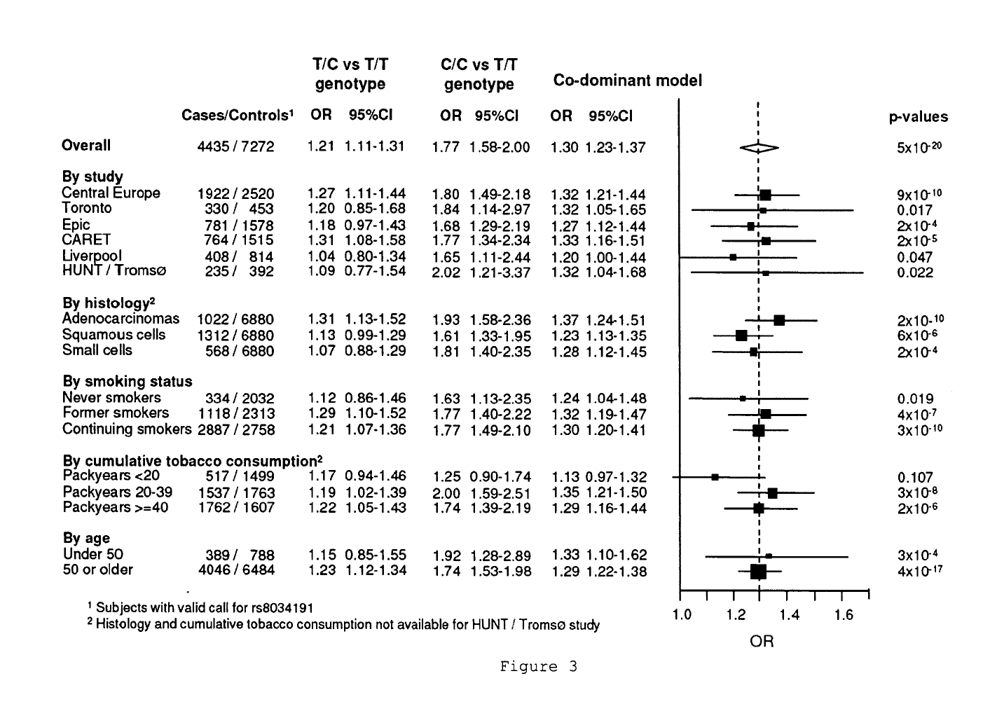

The FIG. 3 shows the OR and 95% CI for lung cancer comparing heterozygous (T/C) and homozygous (C/C) genotypes of rs8034191 to homozygous (T/T) genotype. The ORs are standardized by age, sex and country, and the overall OR is shown by the broken vertical line. Finally, the P-values are derived from the co-dominant model.

The results established that a similar increased risk is observed for both heterozygous and homozygous variants of rs8034191 in all 5 replication studies to that observed in the central Europe study (see FIG. 3). After pooling across all 6 studies, the ORs and 95% CIs were 1.21 (1.11-1.31) and 1.77 (1.58-2.00) for heterozygous and homozygous carriers respectively, the allelic OR was 1.30 (1.23-1.37) and the p-value for trend was 5×10⁻²⁰. Adjustment for cumulative tobacco consumption (packyears) had little effect on these estimates (allelic OR=1.26, 1.18-1.34). There was no statistical evidence of heterogeneity in the increase in risk across the 6 studies and there were no major differences between the main histological subtypes of lung cancer. An increased risk was apparent for never smokers, former smokers and current smokers. A similar risk was observed after stratifying by age at diagnosis and smoking intensity (see FIG. 3). The prevalence of the variant allele was 34% resulting in 66% of the control participants carrying at least one copy, and the percentage of lung cancer explained by carrying at least one allele (i.e. the population attributable risk) was 16%.

### 5) Association of 15q25 Markers with Lung Cancer Death

It has been previously estimated that the cumulative risk of lung cancer death among men in the 6 central European countries to be about 1% in never smokers, 5% among men who have quit smoking and 16% among continuing smokers (BRENNAN et al., Am. J. Epi., vol. 164, p: 1233-1241, 2006).

To illustrate the strong impact of the 15q25 locus, we have calculated cumulative risks for different genotypes in smokers and former smokers compared to non-smokers, using Polish men as a representative example. These calculations of cumulative risk have involved combining national lung cancer mortality rates with the relative risks associated with various smoking patterns, and with the prevalence of those patterns among controls (BRENNAN et al., abovementioned, 2006).

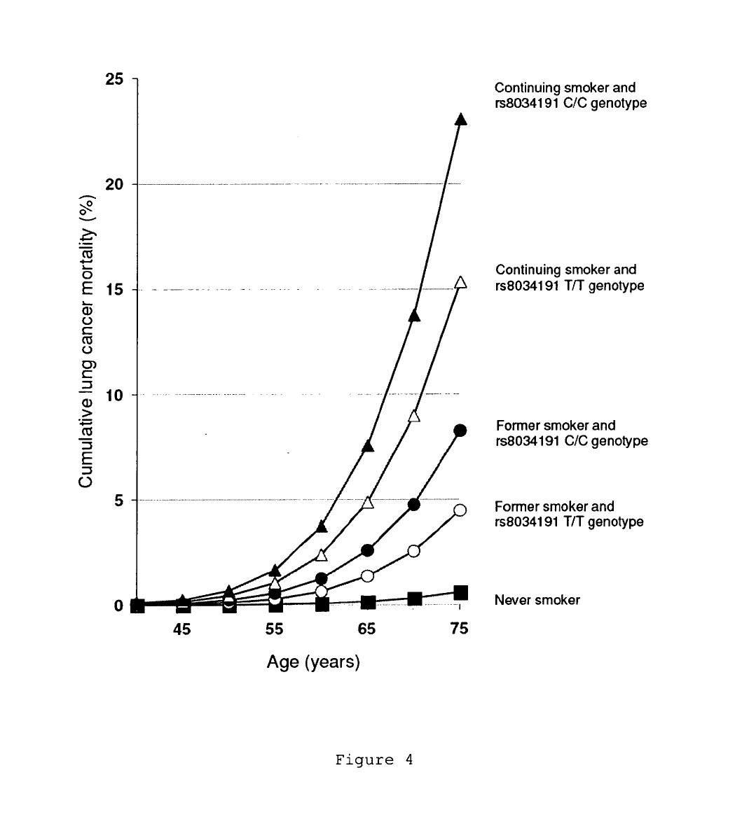

The FIG. 4 shows the relevance of smoking and of rs8034191 genotype to lung cancer mortality in men aged 45-75 years. Cumulative risk (in the absence of other causes of death) based on national lung cancer death rates for men in Poland in the year 2000, assuming that the prevalence of smoking, former smoking, and never smoking are as in this study and that the relative risks for lung cancer incidence and mortality are similar.

The results show that the cumulative risks of lung cancer death by age 75 are about 14% and 23% among smokers with the common and minor homozygous genotype respectively (see FIG. 4). Similarly, among former smokers the cumulative risks are about 4% and 8% respectively. Interestingly, the risk haplotype is rare in the Asian (Japanese and Chinese) and not observed in African (Yoruba) data in the HapMap database and many of the risk alleles have varied allele frequencies (see Table II), which implicate a different contribution of the 15q25 locus to lung cancer risk in these ethnic groups.

### 6) Association of 15q25 Markers with Lung Cancer is Specific

It has been further investigated whether the locus was associated with cancers of the head and neck including those of the oral cavity, larynx, pharynx and esophagus.

For this purpose, rs8034191 was analyzed in two separate studies of head and neck cancer conducted in Europe, the first being conducted in 5 countries of central Europe (i.e., Russia, Czech Republic, Romania, Hungary and Poland) and overlapping with the lung cancer controls from 5 of the 6 countries included in the present genome-wide association study (726 cases including 238 oral cavity/pharynx, 312 larynx and 156 esophagus and 20 with an overlapping site, and 694 controls) and the second study being conducted in 8 countries of Europe (the ARCAGE study) and including 1536 cases (749 oral cavity/pharynx, 574 larynx, 159 esophagus and 54 cases with an overlapping site) and 1443 controls.

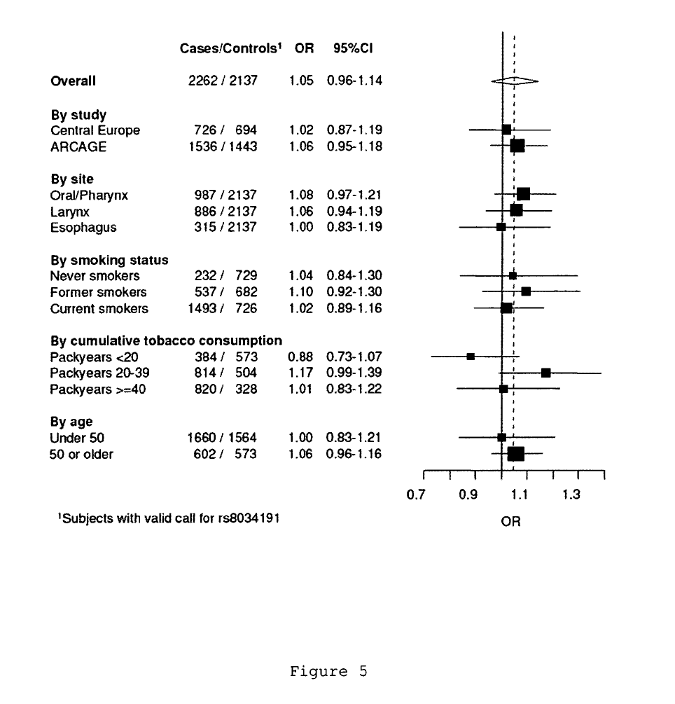

The FIG. 5 shows the OR and 95% CI for upper aero-digestive cancer for the rs8034191 variant using the co-dominant model among 2,262 cases and 2,137 controls. The ORs are standardized by age, sex and country.

The results show that no effect with rs8034191 was observed in either of the two studies separately or combined or in any of the cancer subgroups (See FIG. 5), implying that this association was specific for lung cancer.

### 7) Identity of the Gene(s) Associated with Lung Cancer Predisposition

The present study has identified markers associated with lung cancer, said disease-associated markers spanning six known genes, including the nicotinic acetylcholine receptor subunits CHRNA5, CHRNA3 and CHRNB4, the IREB2 iron sensing response element, PSMA4, which is implicated in DNA repair, and a gene of unknown function designated LOC123688 (see FIG. 2).

It was not possible to identify the most likely causal alleles or genes based on the differences in the strength of the statistical association because of the strong linkage disequilibrium.

However, the nicotinic acetylcholine receptor subunits are strong candidate genes. CHRNA5 was the only gene found to contain a non-synonymous variant (rs16969968 in exon 5) with a strong disease association (p_{trend}=1.4×10⁻¹⁰). CHRNA3 contained a synonymous variant in exon 5 of CHRNA3 (rs1051730) that was also strongly associated with disease (P_{trend}=3×10⁻¹⁰).

While the other markers with strong disease-association either resided in introns or were inter-genic, it cannot be excluded that they could have a biological effect on one or more of the genes from the region.

However, other lines of evidence support a possible role for the nicotinic acetylcholine receptor subunit genes.

CHRNA5, CHRNA3 and CHRNB4 are part of a large family of nicotinic acetylcholine receptor subunit. These genes encode proteins forming receptors present in neuronal and other tissues that bind nicotine and nicotine derivatives. An association of CHRNA3 and CHRNA5 variants with nicotine dependence has been reported (SACCONE et al., abovementioned, 2007). The associated markers include the non-synonymous CHRNA5 SNP, rs16969968, which is one of the markers of lung cancer risk. This SNP introduces a substitution of aspartic acid (D) to asparagine (N) at amino acid position 398 (D398N) of the CHRNA5 protein, located in the central part of the second intracellular loop. While the function of the second intracellular loop and the possible biological consequences of the D398N alteration, creating a charge change, remain to be elucidated, this amino acid is highly conserved across species suggesting that it could have functional importance. A T529A substitution in the second intracellular loop of CHRNA4, another nicotinic aceltycholine receptor subunit, is known to lead to altered responses to nicotine exposure in the mouse.

Within the ARCAGE study, all participants were asked a series of questions relating to tobacco addiction based on the FAGERSTROM tolerance questionnaire. No association between the genotyped marker of lung cancer risk, rs8034191, and any of the FAGERSTROM measures of nicotine addiction was observed, and similar results were observed for rs16969968.

Consequently, nicotinic acetylcholine receptors may be involved in lung cancer through mechanisms other than tobacco dependence.

Finally, the expression of nicotine acetylcholine receptors can be inhibited by nicotine receptor antagonists that, if confirmed to be involved in disease etiology through such a mechanism, imply possible chemoprevention opportunities for lung cancer.

1.-9. (canceled)

10. A method of testing a human thought to be predisposed to having lung cancer which comprises the step of i) analyzing a biological sample from said human for detecting the presence of a polymorphism on chromosome 15q25 associated with lung cancer.

11. The method according to claim 10, wherein the detection of the presence of a polymorphism on chromosome 15q25 associated with lung cancer in said human enables to confirm that said human has or is predisposed for having cancer.

12. The method according to claim 10, wherein said polymorphism on chromosome 15q25 corresponds to a polymorphism located between position 76,480,000 and 76,800,000 of the human chromosome 15 (NC_{—}000015).

13. The method according to claim 10, wherein said polymorphism on chromosome 15q25 corresponds to a polymorphism located between position 76,499,754 and 76,795,005.

14. The method according to claim 10, wherein said biological sample is a blood sample.

15. The method according to claim 10, wherein said polymorphism on chromosome 15q25 associated with lung cancer corresponds to a single nucleotide polymorphism (SNP).

16. The method according to claim 15, wherein said single nucleotide polymorphism (SNP) on chromosome 15q25 associated with lung cancer is selected from the group comprising rs17483548 (nucleotide N at position 16 of SEQ ID NO:1, wherein allele A is associated to lung cancer), rs17405217 (nucleotide N at position 16 of SEQ ID NO:2, wherein allele T is associated to lung cancer), rs17483721 (nucleotide N at position 16 of SEQ ID NO:3, wherein allele C is associated to lung cancer), rs2656052 (nucleotide N at position 16 of SEQ ID NO:4, wherein allele G is associated to lung cancer), rs2568494 (nucleotide N at position 16 of SEQ ID NO:5, wherein allele A is associated to lung cancer), rs7181486 (nucleotide N at position 16 of SEQ ID NO:6, wherein allele C is associated to lung cancer), rs17483929 (nucleotide N at position 16 of SEQ ID NO:7, wherein allele A is associated to lung cancer), rs2656065 (nucleotide N at position 16 of SEQ ID NO:8, wherein allele T is associated to lung cancer), rs2009746 (nucleotide N at position 16 of SEQ ID NO:9, wherein allele C is associated to lung cancer), rs17484235 (nucleotide N at position 16 of SEQ ID NO:10, wherein allele G is associated to lung cancer), rs1504550 (nucleotide N at position 16 of SEQ ID NO:11, wherein allele C is associated to lung cancer), rs17484524 (nucleotide N at position 16 of SEQ ID NO:12, wherein allele G is associated to lung cancer), rs9788721 (nucleotide N at position 16 of SEQ ID NO:13, wherein allele C is associated to lung cancer), rs8034191 (nucleotide N at position 16 of SEQ ID NO:14, wherein allele C is associated to lung cancer), rs10519203 (nucleotide N at position 16 of SEQ ID NO:15, wherein allele G is associated to lung cancer), rs8031948 (nucleotide N at position 16 of SEQ ID NO:16, wherein allele T is associated to lung cancer), rs931794 (nucleotide N at position 31 of SEQ ID NO:17, wherein allele G is associated to lung cancer), rs2036527 (nucleotide N at position 31 of SEQ ID NO:18, wherein allele A is associated to lung cancer), rs17486278 (nucleotide N at position 16 of SEQ ID NO:19, wherein allele C is associated to lung cancer), rs7180002 (nucleotide N at position 16 of SEQ ID NO:20, wherein allele T is associated to lung cancer), rs951266 (nucleotide N at position 16 of SEQ ID NO:21, wherein allele T is associated to lung cancer), rs16969968 (nucleotide N at position 16 of SEQ ID NO:22, wherein allele A is associated to lung cancer), rs1051730 (nucleotide N at position 31 of SEQ ID NO:23, wherein allele A is associated to lung cancer), rs1317286 (nucleotide N at position 16 of SEQ ID NO:24, wherein allele G is associated to lung cancer), and rs17487223 (nucleotide N at position 16 of SEQ ID NO:25, wherein allele T is associated to lung cancer).

17. The method according to claim 16, wherein said single nucleotide polymorphism on chromosome 15q25 associated with lung cancer is selected from the group comprising rs2656052 (nucleotide N at position 16 of SEQ ID NO:4, wherein allele G is associated to lung cancer), rs17484235 (nucleotide N at position 16 of SEQ ID NO:10, wherein allele G is associated to lung cancer), rs8034191 (nucleotide N at position 16 of SEQ ID NO:14, wherein allele C is associated to lung cancer), rs10519203 (nucleotide N at position 16 of SEQ ID NO:15, wherein allele G is associated to lung cancer), rs8031948 (nucleotide N at position 16 of SEQ ID NO:16, wherein allele T is associated to lung cancer), rs931794 (nucleotide N at position 31 of SEQ ID NO:17, wherein allele G is associated to lung cancer), rs2036527 (nucleotide N at position 31 of SEQ ID NO:18, wherein allele A is associated to lung cancer), rs16969968 (nucleotide N at position 16 of SEQ ID NO:22, wherein allele A is associated to lung cancer), and rs1317286 (nucleotide N at position 16 of SEQ ID NO:24, wherein allele G is associated to lung cancer).

18. The method of claim 10, wherein said method further comprises the step of ii) determining the cumulative risks for said human to be predisposed to having a lung cancer by further determining the smoking pattern of said human.

19. A method of identifying a potential modulator of lung cancer, wherein said method comprises the steps of:
contacting a putative potential modulator to a protein selected in the group consisting of: IREB2, such as SEQ ID NO: 33, LOC123688, such as SEQ ID NO: 34 and SEQ ID NO:35, PSMA4, such as SEQ ID NO: 36, CHRNA5, such as SEQ ID NO: 37, CHRNA3, such as SEQ ID NO: 38, and CHRNB4, such as SEQ ID NO: 39, or to a nucleic acid that encodes such a protein; and
monitoring for an effect of the putative potential modulator on the protein or nucleic acid that encodes such protein, thereby identifying whether the putative potential modulator modulates the protein or the nucleic acid that encodes such protein and is therefore a potential modulator of lung cancer.

20. The method according to claim 11, wherein said polymorphism on chromosome 15q25 corresponds to a polymorphism located between position 76,480,000 and 76,800,000 of the human chromosome 15 (NC_{—}000015).

21. The method according to claim 11, wherein said polymorphism on chromosome 15q25 corresponds to a polymorphism located between position 76,499,754 and 76,795,005.
